(19) United States
(12) Patent Application Publication
(10) Pub. No.: US 2026/0265684 A1
(43) Pub. Date: Sep. 10, 2026

(54) EIMERIA VACCINE

(71) Applicant: Eimeria Pty Ltd., Ringwood (AU)

(72) Inventors: Vladimir Vrba, Victoria (AU); Sarika Kosaraju, Victoria (AU)

(21) Appl. No.: 19/662,380

(22) Filed: Apr. 29, 2026

Related U.S. Application Data
(63) Continuation of application No. PCT/AU2025/050229, filed on Mar. 13, 2025.

(30) Foreign Application Priority Data
Mar. 13, 2024 (AU) 2024900659

(51) Int. Cl. C12N 3/00; A61K 9/00; A61K 39/00; A61K 39/002; A61P 31/00

(52) U.S. Cl. CPC C12N 3/00; A61K 9/0048; A61K 39/002; A61P 31/00; A61K 2039/54; A61K 2039/545; A61K 2039/552

(57) ABSTRACT

The present disclosure relates to methods and compositions for the production of oocysts from protozoa, such as Eimeria. The present disclosure also relates compositions comprising the oocysts and use of these composition for the treatment and/or prevention of infections.

This application claims priority from Australian application no. 2024900659 filed Mar. 13, 2024, the entire contents of which are incorporated by reference herein.

### TECHNICAL FIELD

The present disclosure relates to methods and compositions for the production of oocysts from protozoa; such as Eimeria. The present disclosure also relates compositions comprising the oocysts and use of these composition for the treatment and/or prevention of infections.

### BACKGROUND

Coccidiosis is one of the highest mortality-causing diseases in the poultry industry. It is caused by an intestinal protozoan parasite belonging to the genus Eimeria. The infective form of Eimeria is a highly resistant oocyst, shed in the faeces of chickens. The oocysts can persist in the environment for a long time but are only infective once sporulated. Sporulation is an important step in the life cycle of Eimeria and typically requires exposure of the oocyst to adequate moisture, air and warmth. The intensive nature of poultry production, in which thousands of birds are often accumulated in a limited closed space, requires an efficient prevention and control of coccidiosis. Control depends on decreasing environmental exposure (through decontamination) and the use of anticoccidials. However, in many cases, coccidiosis cannot be controlled effectively even if anticoccidial drugs are used correctly.

An alternative method for control of coccidiosis is vaccination, often using a live vaccine to provide a unform exposure to the protozoa. Eimeria vaccines are produced from oocysts which are harvested from chicken faeces. Chicken faeces are heavily laden with bio-burden and the oocysts need to be sporulated and sterilised before use as a vaccine component. Achieving sterility of the final product is one of the main challenges in the manufacture. Currently, the most common method for manufacture of live Eimeria vaccines uses 2% potassium dichromate as the sporulation medium and sodium hypochlorite for sterilisation, which is a strong oxidising agent with anti-microbial properties. However, potassium dichromate is highly toxic and detrimental to the environment. Due to these factors it is not widely accepted globally in vaccine products. In addition, the current manufacturing process includes a large number of steps, requires intensive manual handling and has been difficult to automate and scale up.

With an increasing demand for the vaccine and the push to scale up production, there remains a need for improved methods of producing sporulated and sterile oocysts for use in future vaccines.

### SUMMARY

The inventors of the present application have surprisingly found that low levels of a peroxycarboxylic acid, such as peracetic acid, can be used to sporulate protozoan oocysts and/or sterilise a composition comprising protozoan oocysts. The inventors have also found that methods of vaccine manufacture using peroxycarboxylic acid for both sporulation and sterilisation may be performed in a single vessel, which helps streamline the process. Accordingly, described herein are improved methods and compositions for producing protozoan oocysts (e.g., Eimeria), for example, for use in the manufacture of vaccines. These methods provide one or more advantages over the traditional methods as they allow for less manual handling, reduce the need for toxic chemicals, can be performed in a single vessel, can be automated and/or easily scaled up.

Provided herein is a method for preparing a formulation comprising viable Eimeria oocysts, the method comprising:
incubating viable oocysts with a sporulating composition comprising a first concentration of peroxycarboxylic acid to produce viable, sporulated Eimeria oocysts; and
incubating the viable, sporulated Eimeria oocysts with a sterilising composition comprising a second concentration of the peroxycarboxylic acid to produce the formulation,
wherein the second concentration of peroxycarboxylic is greater than the first concentration.

In some examples, the incubating is performed in a single reaction vessel. In some examples, the second concentration is the concentration of the peroxycarboxylic acid added to the single reaction vessel to initiate the sterilisation step.

In some examples, first concentration of peroxycarboxylic acid is less than 20,000 ppm, or less than 7200 ppm, or less than 5,000 ppm, or less than 1,000 ppm, less than 750 ppm less than 500 ppm, or less than about 300 ppm. In some examples, first concentration of peroxycarboxylic acid is between 250 and 350 ppm.

In some examples, the second concentration of peroxycarboxylic acid is >about 250 ppm, ≥about 500 ppm, ≥ about 750 ppm or >about 1,000 ppm.

In some examples, the total concentration of peroxycarboxylic acid added during the method is the sum of the first and second concentrations. In some examples, the total concentration is >about 1250 ppm, ≥about 1350 ppm, ≥ about 1400 ppm. In some examples, the total concentration is between about 1000 ppm and 2000 ppm.

In some examples, the peroxycarboxylic acid is a C₂-C₅ alkyl peroxycarboxylic acid. In some examples, the peroxycarboxylic acid is peracetic acid.

In some examples, the oocysts are incubated with the sporulating composition at a solids content of between about 0.05% v/v and 10% v/v, or between about 1% v/v and 6% v/v, or between about 4.5% v/v and 5.5% v/v. In some examples, the oocysts are incubated with the sterilising composition at a solids content of between about 0.05% v/v and 10% v/v, or between about 1% v/v and 6% v/v, or between about 4.5% v/v and 5.5% v/v.

In some examples, the method further comprises: processing the sterilised formulation to substantially remove the peroxycarboxylic acid. In some examples, processing the sterilised formulation to substantially remove the peroxycarboxylic acid comprises buffer exchange, for example diafiltering the sterilised formulation by one or more steps of tangential flow filtration (TFF). In some examples, the at least one or more steps of TFF comprises using a hollow fiber membrane cartridge.

In some examples, the formulation is a sterilised formulation. In some examples, the formulation does not comprise detectable amounts of Staphylococcus aureus, Pseudomonas aeruginosa, Bacillus subtilis, Candida albicans, Aspergillus brasiliensis, Clostridium sporogenes, Paenibacillus rigui and/or Paenibacillus glucanolyticus, for example as assessed using the assays exemplified herein.

As disclosed herein, there is also provided a composition comprising a sterilised formulation produced by the method described herein. In some examples, the composition comprises viable Eimeria oocysts for at least 12, 18, 24, 39, 36, 42 or 48 weeks after storage at 4-8° C.

As disclosed herein, there is also provided a pharmaceutical composition comprising a sterilised formulation produced by the method described herein and a pharmaceutically acceptable carrier. In some examples, the composition comprises viable Eimeria oocysts for at least 12, 18, 24, 39, 36, 42 or 48 weeks after storage at 4-8° C. In some examples, the composition is a vaccine composition. In some examples, the vaccine composition comprises a peroxycarboxylic acid as described herein. In some examples, the composition comprises peracetic acid. In some examples, the composition comprises less than about 100 ppm peracetic acid. In some examples, the composition comprises between about 10 ppm and about 100 ppm peracetic acid.

As disclosed herein, there is also provided a method of immunizing poultry against Eimeria infection comprising administering to the poultry the vaccine composition described herein.

As disclosed herein, there is also provided a method of enhancing the immune response against an Eimeria parasite in poultry comprising administering the vaccine composition as described herein in an amount effective to enhance the immune response of the poultry to the Eimeria parasite.

As disclosed herein, there is also provided a method of treating or preventing coccidiosis in a poultry, comprising administering to the poultry the vaccine composition as described herein in an amount effective to treat or prevent coccidiosis.

As disclosed herein, there is also provided a method for sporulating viable Eimeria oocysts comprising:
incubating the oocysts with a sporulating composition comprising a first concentration of peroxycarboxylic acid to produce viable, sporulated Eimeria oocysts,
wherein the first concentration of peroxycarboxylic acid comprises less than 20,000 ppm peroxycarboxylic acid.

In some examples, the least one peroxycarboxylic acid is a C₂-C₅ alkyl peroxycarboxylic acid. In some examples, the at least one peroxycarboxylic acid is peracetic acid. In some examples, the concentration of the peroxycarboxylic acid is between about 200 and 300 ppm. In some examples, the sporulating composition comprises hydrogen peroxide at a concentration of about less than about 5000 ppm. In some examples, the weight ratio of peroxycarboxylic acid to hydrogen peroxide is from about 1:1 to about 1:10.

In some examples, the oocysts are incubated with the sterilising composition at a solids content of between about 0.05% v/v and 10% v/v, or between about 1% v/v and 6% v/v, or between about 4.5% v/v and 5.5% v/v.

In some examples, the sterilising and/or sporulation composition further comprises a detergent and/or antifoaming agent. In some examples, the detergent is docusate, optionally 0.25% (w/v) docusate.

In some examples, the pH of the sterilising and/or sporulation composition is less than 7.0, for example between 2.0 and 4.0.

In some examples, the oocysts are incubated with the sporulating composition for between about 24 to 72 hrs, or about 40 to 48 hrs. In some examples, the oocysts are incubated with the sporulating composition at a temperature of between about 20 and 32° C., or about 27° C.,

In some examples, the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof.

As disclosed herein, there is also provided a composition comprising Eimeria oocysts and peroxycarboxylic acid at a concentration of less than 20,000 ppm, for example between 1 ppm and 1500 ppm peroxycarboxylic acid. In some examples, the at least one peroxycarboxylic acid is peracetic acid. In some examples, the concentration of the peroxycarboxylic acid is from 50 ppm to about 300 ppm. In some examples, the concentration of the peroxycarboxylic acid is from 1 ppm to about 300 ppm. In some examples, the concentration of the peroxycarboxylic acid is from 10 ppm to about 100 ppm. In some examples, the concentration of the peroxycarboxylic acid is from 800 ppm to about 1200 ppm. In some examples, the composition further comprises a buffer, such as phosphate buffered saline. In some examples, the composition further comprises a detergent, such as Tween 80. In some examples, the composition is a vaccine composition.

In some examples, the composition has a solids content about 0.05% v/v and 10% v/v, or between about 1% v/v and 6% v/v, or between about 4.5% v/v and 5.5% v/v.

In some examples, the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof. In some examples, the Eimeria oocysts comprise unsporulated oocysts. In some examples, the Eimeria oocysts comprise sporulated oocysts.

As disclosed herein, there is also provided a composition comprising a population of sporulated Eimeria oocysts, wherein the composition is sterile and, wherein the sporulated oocysts have been treated with a first concentration of peroxycarboxylic acid and a second concentration of peroxycarboxylic acid in a single vessel, wherein the first concentration of peroxycarboxylic acid is less 1000 ppm and the second concentration of peroxycarboxylic increased relative to the first concentration.

In some examples, the at least one peroxycarboxylic acid is peracetic acid. In some examples, first concentration of peroxycarboxylic acid is from about 40 ppm to about 300 ppm, and/or wherein the second concentration of peroxycarboxylic acid is from about 800 ppm to about 1200 ppm. In some examples, the composition has a solids content about 0.05% v/v and 10% v/v, or between about 1% v/v and 6% v/v, or between about 4.5% v/v and 5.5% v/v. In some examples, the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof.

In some examples, the composition is further processed to substantially remove the peroxycarboxylic acid, preferably wherein the composition is diafiltered using TFF.

In some examples, the composition comprises viable oocysts for at least 12, 18, 24, 30, 36, 42 or 48 weeks after storage at 4-8° C. In some examples, the composition is sterile.

### BRIEF DESCRIPTION OF DRAWINGS

The accompanying drawings, which constitute a part of this specification, illustrate several exemplary embodiments of the disclosure and together with the description, serve to explain the principles of the disclosure. The embodiments disclosed in the drawings are exemplary and do not limit the scope of this disclosure.

FIG. 1—illustrates the percentage sporulation of E. acervulina oocysts for each of the sporulation media tested in Example 1. The results of three replicates are shown.

FIG. 2—illustrates the percentage sporulation of E. brunetti oocysts for each of the sporulation media tested in Example 1. The results of three replicates are shown.

FIG. 3—illustrates the percentage sporulation of E. maxima oocysts for each of the sporulation media tested in Example 1. The results of three replicates are shown.

FIG. 4—illustrates the percentage sporulation of E. mitis oocysts for each of the sporulation media tested in Example 1. The results of three replicates are shown.

FIG. 5—illustrates the percentage sporulation of E. necatrix oocysts for each of the sporulation media tested in Example 1. The results of three replicates are shown.

FIG. 6—illustrates the percentage sporulation of E. tenella oocysts for each of the sporulation media tested in Example 1. The results of three replicates are shown.

FIG. 7—illustrates the averaged sporulation of E. acervulina oocysts from the three replicate experiments for each sporulation media tested in Example 1. Also shown is the difference n percent sporulation compared to 2% potassium dichromate.

FIG. 8—illustrates the averaged sporulation of E. brunetti oocysts from the three replicate experiments for each sporulation media tested in Example 1. Also shown is the difference n percent sporulation compared to 2% potassium dichromate.

FIG. 9—illustrates the averaged sporulation of E. maxima oocysts from the three replicate experiments for each sporulation media tested in Example 1. Also shown is the difference n percent sporulation compared to 2% potassium dichromate.

FIG. 10—illustrates the averaged sporulation of E. mitis oocysts from the three replicate experiments for each sporulation media tested in Example 1. Also shown is the difference n percent sporulation compared to 2% potassium dichromate.

FIG. 11—illustrates the averaged sporulation of E. necatrix oocysts from the three replicate experiments for each sporulation media tested in Example 1. Also shown is the difference n percent sporulation compared to 2% potassium dichromate.

FIG. 12—illustrates the averaged sporulation of E. tenella oocysts from the three replicate experiments for each sporulation media tested in Example 1. Also shown is the difference n percent sporulation compared to 2% potassium dichromate.

FIG. 13—illustrates the results of the sterility experiments for each sporulation media tested.

FIG. 14—illustrates the relationship between percent solids content and sporulation for 2% potassium dichromate, 2% Proxitane (~1000 ppm peracetic acid), 0.1% Proxitane (~50 ppm peracetic acid), 2% potassium permanganate and 0.5% hydrogen peroxide.

FIG. 15—illustrates the relationship between percent solids content and sporulation for 2% potassium dichromate, 2% Proxitane (~1000 ppm peracetic acid), 0.1% Proxitane (~50 ppm peracetic acid), 2% potassium permanganate and 0.5% hydrogen peroxide.

FIG. 16—illustrates the turbidity of a composition comprising 1% solids for each of E. mitis, E. maxima, E. acervulina, E. brunetti, E. tenella and E. necatrix. Three replicates are shown for E. mitis, E. maxima, E. acervulina, and E. brunetti. Two replicates are shown for E. tenella and E. necatrix.

FIG. 17—illustrates a comparison of percentage sporulation verses turbidity for each sample tested.

FIG. 18—illustrates a time course of the chemical inactivation of different bacterial and fungal species using (A) 0.5% hydrogen peroxide, (B) 0.1% Proxitane (~50 ppm peracetic acid) and (C) 2% Proxitane (~1000 ppm peracetic acid).

FIG. 19—illustrates the effect of different detergents on the percentage sporulation of E. mitis.

FIG. 20—illustrates the effect of 0.25% docusate on the percentage sporulation of different Eimeria species.

FIG. 21—illustrates the overall RPT results (oocyst count oo/bird/day) over time for the MMAT. Two groups are shown for the MMAT vaccine.

FIG. 22—illustrates the RPT results (oocyst count oo/bird/day) over time for the four different Eimeria species in the MMAT vaccine. The MMAT vaccine includes (A) E. acervulina, (B) E. maxima, (C) E. mitis and (D) E. tenella. Two groups are shown.

### DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive). Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, biochemistry, mass spectrometry, analytical chemistry and separation science).

Unless otherwise indicated, the chemistry, biochemistry, microbiology, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

As used herein, the term “approximately” or “about,” as applied to one or more values of interest, refers to a value that is similar to a stated reference value. In some examples, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some examples, the term “approximately” or “about” refers to a range of values that fall within about ±10% of the stated reference value unless otherwise stated or otherwise evident from the context. In some examples, the term “approximately” or “about” refers to a range of values that fall within about +5% of the stated reference value unless otherwise stated or otherwise evident from the context.

Throughout the present specification, various aspects and components of the disclosure can be presented in a range format. The range format is included for convenience and should not be interpreted as an inflexible limitation on the scope of the disclosure. Accordingly, the description of a range should be considered to have specifically disclosed all the possible subranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

### Selected Definitions

As used herein, the terms “subject” and “animal subjects,” include but are not limited to, mammalian and avian subjects, preferably avian subjects. Suitable mammalian subjects include but are not limited to human, simian, porcine, bovine, caprine, equine, feline, ovine, canine, murine and lagomorph subjects.

As used herein, the terms “avian” and “avian subjects” or “bird” and “bird subjects”, are intended to include males and females of any avian or bird species, but are primarily intended to encompass poultry which are commercially raised for eggs, meat or as pets. Accordingly, in some examples the terms “avian” and “avian subject” or “bird” and “bird subject” comprise poultry. In some examples the terms “avian” and “avian subject” or “bird” and “bird subject” comprise chickens, turkeys, ducks, geese, quail, pheasant, parakeets, parrots, cockatoo, cockatiel, ostrich, emu and the like. Chickens and turkeys are the preferred avian or bird subjects, with chickens being most preferred.

As used herein (and in the context of Eimeria preparations suitable for use as vaccines), the terms “microbial contamination” or “contamination by microorganisms” or “bio-burden” are intended to indicate the presence of detectable and unwanted viable microorganisms including but not limited to bacteria, molds, fungi, yeast and viruses. In some examples, the oocyst preparation (e.g. produced using the methods described herein) is essentially free of detectable microbial contamination, meaning that no significant levels of microbial contamination are detected in the preparation. In some examples, the levels of microbial contamination is below the threshold required for vaccine products.

As used herein, the terms “disease”, “disorder” or “condition” refers to a disruption of or interference with normal function, and is not to be limited to any specific condition, and will include diseases or disorders.

As used herein, a subject “at risk” of developing a disease, disorder or condition may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering an RNA or composition described herein to thereby reduce or eliminate at least one symptom of a specified disease or condition.

As used herein, the term “preventing”, “prevent” or “prevention” includes providing prophylaxis with respect to occurrence or recurrence of a specified disease or condition in an individual. An individual may be predisposed to or at risk of developing the disease but has not yet been diagnosed with the disease.

As used herein, the phrase “delaying progression of” includes reducing or slowing down the progression of the disease or condition in an individual and/or at least one symptom of a disease or condition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

An “effective amount” refers to at least an amount effective, at dosages and for periods of time necessary, to achieve the desired result. For example, the desired result may be a therapeutic or prophylactic result. In some examples of the present disclosure, the term “effective amount” or “therapeutically effective amount” of a sporulated oocyst is an amount sufficient to produce the desired effect, such as an immune response and/or infection in an animal treated with the oocyst. Suitable assays for an immune response and/or infection are known to the person skilled in the art. The effective amount may vary according to the Eimeria species and also according to the weight, age, sex, health and/or physical condition and other factors relevant to the animal being treated. Typically, the effective amount will fall within a relatively broad range (e.g. a “dosage” range) that can be determined through routine trial and experimentation by a veterinary practitioner. Accordingly, this term is not to be construed to limit the disclosure to a specific quantity, e.g., weight or number of oocysts. An effective amount can be provided in one or more administrations. For example, the effective amount can be administered in a single dose or in a dose repeated once or several times over a treatment period. An effective dose may be acquired after repeated infections resulting from the first vaccination.

A “prophylactically effective amount” shall be taken to mean a sufficient quantity of the sporulated oocysts of the disclosure to prevent or inhibit or delay the onset of one or more detectable symptoms of a disease or disorder as described herein.

As used herein, “viable oocyst” refers to an oocyst that is capable of infecting a subject. As would be understood by the person skilled in the art in the context of a population of oocysts, it is not required that every oocyst in the population of oocysts be viable, just that there are sufficient viable oocysts in the population to infect a subject.

### Oocysts

Many protozoa form a life stage designated as an “oocyst”. The present disclosure relates generally to methods and compositions for the production of oocysts from protozoa. In some examples, the oocysts are suitable for use in vaccines, for example, Coccidiosis vaccines. The methods described herein can be used to produce viable oocysts from any species of protozoa, including but not limited to Eimeria, Cryptosporidium, Toxoplasma, Plasmodia and Isospora. In some examples, the methods are used to produce viable Eimeria oocysts. The terms “protozoa”, “oocyst”, “sporocyst”, “sporozoite” and “merozoite” have their accepted meaning in the art. Unless indicated otherwise, these terms are intended to refer to live protozoa, oocysts, sporocysts, sporozoites and merozoites, although those skilled in the art will appreciate that vaccines may be formulated using killed (or attenuated) protozoa, oocysts, sporocysts, sporozoites and merozoites.

As used herein, the term “Eimeria” means one or more species of the genus Eimeria. In some examples, the term “Eimeria” comprises strains or species of Eimeria that infect avian or mammalian species. In some examples, Eimeria species comprise those that infect poultry, such as chickens, turkeys, geese, ducks, guinea fowl and squabs. In some examples, Eimeria species comprise those that infect sheep, goats or cattle. In some examples, Eimeria species comprise those that infect chickens or turkeys. In some examples, Eimeria species comprise those that infect chickens. Eimeria species found in chickens include, but are not limited to, E. tenella, E. acervulina, E. maxima, E. necatrix, E. mitis, E. praecox, E. mivati, E. lata, E. nagambie, E. zaria and E. brunetii. Eimeria species found in turkeys include, but are not limited to, E meleagrimitis, E. adenoeides, E. gallopavonis, E. dispersa, E. innocua, and E. subrotunda. Eimeria species found in goats include, but are not limited to, E. ninakohlyakimovae, E. arloingi, E. caprina, E. christenseni, E. parva, E. hirci, E. jolchijevi, E. apsheronica, E. alijevi, E. caprovina, E. capralis, E. charlestoni, E. masseyensis, E. pallida and E. punctata, preferably E. ninakohlyakimovae, E. arloingi, E. caprina and E. christenseni. Eimeria species found in sheep include, but are not limited to, E. ahsata, E bakuensis, E crandallis, E faurei, E gilruthi (previously Globidium gilruthi), E granulosa, E intricata, E marsica, E ovina (E arloingi A), E ovinoidalis (E ninokohlyakimovae), E pallida, E parva, and E weybridgensis (E arloingi B), preferably E crandallis and E ovinoidalis.

In addition, the term “Eimeria” includes all strains of the foregoing species of Eimeria, including but not limited to wildtype strains, precocious or otherwise selected strains, attenuated strains, and oocysts that have been attenuated, e.g., by irradiation, chemical treatment and the like. Further, the term “Eimeria” also includes any newly-discovered strains or species of Eimeria. Finally, the term “Eimeria” encompasses live and killed Eimeria, although live Eimeria are intended unless indicated otherwise.

Any suitable method of obtaining oocysts known to the person skilled in the art may be used to obtain oocysts for use in the methods described herein. Oocysts are obtainable from faeces or tissue of infected animals; contaminated feed or water; soil; pen litter or bedding; or a variety of other sources. Methods for isolation of sporocysts and oocysts are known. The exact procedures used to separate oocysts will vary with the material from which the oocysts are obtained and will be readily apparent to those skilled in the art. Typically, these methods involve infecting an animal with the protozoan of interest, collecting faeces that contain oocysts from the infected animal, purifying the oocysts from the faecal material through a series of separation procedures (e.g. sieving, centrifugation, filtration and/or density flotation) to obtain the oocysts. In some examples, the oocysts may be obtained from an infected animal (e.g. a bird, such as a chicken). In some examples, the oocysts may be produced by collecting faeces containing oocysts from an infected animal and purifying the oocysts. In some examples, the oocysts may be produced by infecting an animal, collecting faeces containing oocysts therefrom, and purifying the oocysts. Unless indicated otherwise, the terms “purifying”, “purify” “purification” and “purified” and variations thereof are used herein with respect to preparations of oocysts refer to the separation from, or removal of, debris and other unwanted material from preparations containing the oocysts. These terms are intended to indicate that the degree of isolation or separation of the oocysts from other material present in the faeces is enhanced, not that absolutely all extraneous materials are removed from the oocyst preparations. Likewise, the oocyst preparation may contain some degree of microbial contamination, as long as the final preparation is suitable for its intended use (e.g., as a vaccine). In the case of vaccines intended for in ovo administration or eye drop administration to birds, in particular examples, the preparation will be essentially free of detectable contamination by microorganisms, in particular, microorganisms that are pathogenic (i.e., cause significant illness or mortality) to the embryo. Depending upon context, a “purification” process may refer to the entire process of purifying oocysts from faeces to produce a preparation suitable for vaccination purposes. Alternatively, a “purification” process may refer to any subset of steps, or even a single step, within the entire purification scheme.

One available approach to separating the oocysts from extraneous material is as follows. Soil or excreta (e.g. faeces and/or caeca) is generally processed by forming a slurry with saturated saline solution and separating the sporocysts and/or oocysts from the slurry. For example the oocysts may be separated using saturated NaCl flotation. In one example, the material to be processed may be washed with water before being mixed with saturated aqueous NaCl to form a slurry. If necessary, the slurry can be processed in a mixer or blender until a homogenous consistency is achieved. The slurry is centrifuged, the supernatant collected, diluted in water before centrifugation to pellet the oocysts. The pellet is resuspended, washed and used in the subsequent steps. Other methods to purify oocysts from samples that are commonly used include the Sheather sucrose flotation and Zinc-sulfate flotation, [e.g., see L R Ash and T C Orihel, Parasites: A Guide to Laboratory Procedures and Identification, ASCP Press@ 1991, incorporated by reference herein]. After the final wash, the oocysts can be stored, or transferred to a container for sporulation.

### Sporulation

In some examples, the methods described herein comprise sporulation of an oocyst preparation. The oocyst preparation typically comprises viable oocysts which are capable of sporulation under the appropriate conditions. In some examples, the methods described herein comprise incubating viable oocysts with a sporulating composition comprising a first concentration of peroxycarboxylic acid. Sporulation produces viable, sporulated Eimeria oocysts. A peroxycarboxylic acid refers to carboxylic acid having the hydrogen of the hydroxyl group in carboxylic acid replaced by a hydroxy group. A peroxycarboxylic includes any compound of the formula R—(COOOH)ₙ in which R can be hydrogen, alkyl, alkenyl, alkyne, acylic, alicyclic group, aryl, heteroaryl, or heterocyclic group, and n is 1, 2, or 3, and named by prefixing the parent acid with “peroxy” or “per”. In some examples, R comprises hydrogen, alkyl, or alkenyl. In some examples, R comprises hydrogen or alkyl. In some examples, R comprises alkyl. The terms “alkyl,” “alkenyl,” “alkyne,” “acylic,” “alicyclic group,” “aryl,” “heteroaryl,” and “heterocyclic group” are as defined herein.

As used herein, the term “alkyl” encompasses both straight chain (i.e., linear) and branched chain hydrocarbon groups. In some examples, the alkyl group comprises from 1 to 22 carbon atoms (i.e. C₁₋₂₂alkyl). In one example, the alkyl group is of one to six carbon atoms (i.e. C₁₋₆alkyl). Examples of alkyl groups include methyl, ethyl, n-propyl, iso-propyl, n-butyl, t-butyl, i-butyl, sec-butyl, pentyl, and hexyl groups.

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

As used herein, the term “alkenyl” refers to both straight and branched chain unsaturated hydrocarbon groups with at least one carbon-carbon double bond. Examples of alkenyl groups include ethenyl, propenyl, butenyl, pentenyl, and hexenyl groups. In one example, the alkenyl group is of two to 12 carbon atoms (i.e. C₂₋₁₂alkenyl). In one example, the alkenyl group is of two to six carbon atoms (i.e. C₂₋₆alkenyl).

In some examples, the alkyl or alkenyl can be terminally substituted with a heteroatom, such as, for example, a nitrogen, sulfur, or oxygen atom, forming an aminoalkyl, oxyalkyl, or thioalkyl, for example, aminomethyl, thioethyl, oxypropyl, and the like. Similarly, the above alkyl or alkenyl can be interrupted in the chain by a heteroatom forming an alkylaminoalkyl, alkylthioalkyl, or alkoxyalkyl, for example, methylaminoethyl, ethylthiopropyl, methoxymethyl, and the like.

As used herein, the terms “carbocyclyl” and “carbocycle” refer to a monovalent non-aromatic, saturated, or partially unsaturated, or aromatic ring having 3 to 12 carbon atoms (i.e., 3-12 membered carbocylyl) as a monocyclic ring. In one example, the carbocyclyl is a 3-10 membered carbocyclyl. In one example, the carbocyclyl is a 3-8 membered carbocyclyl. In one example, the carbocyclyl is a 3-6 membered carbocyclyl. A carbocyclyl group may, for example, be monocyclic or polycyclic (i.e. bicyclic, tricyclic). A polycyclic carbocyclyl group may contain fused rings. Examples of monocyclic carbocyclyl groups include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, and the like. Examples of monocyclic, aromatic carbocyclyl group include, but are not limited to, phenyl and naphthalenyl.

Further, as used herein the term “alicyclic” includes any cyclic hydrocarbyl which may be either saturated or unsaturated, but does not have aromatic character having, for example, from 3 to 8 carbon atoms. Examples of suitable alicyclic groups include cyclopropanyl, cyclobutanyl, cyclopentanyl, etc. In some examples, the alicyclic group is a cycloalkane or a cycloalkene.

As used herein, the term “heterocyclyl” refers to an aromatic or non-aromatic cyclic group which is analogous to a carbocyclyl group, but in which from one or more of the carbon atoms is/are replaced by one or more heteroatoms independently selected from nitrogen, oxygen, or sulfur. A heterocyclyl group may, for example, be monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). Heterocyclic groups may be saturated or unsaturated. Examples of suitable heterocyclic groups include for example, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan. Additional examples of suitable heterocyclic groups include groups derived from tetrahydrofurans, furans, thiophenes, pyrrolidines, piperidines, pyridines, pyrrols, picoline, coumaline, etc.

In some examples, alkyl, alkenyl, alicyclic groups, and heterocyclic groups can be unsubstituted or substituted by, for example, aryl, heteroaryl, C₁-C₄ alkyl, C₁-C₄ alkenyl, C₁-C₄ alkoxy, amino, carboxy, halo, nitro, cyano, —SO₃H, phosphono, or hydroxy. When alkyl, alkenyl, alicyclic group, or heterocyclic group is substituted, preferably the substitution is C₁-C₄ alkyl, halo, nitro, amido, hydroxy, carboxy, sulpho, or phosphono. In one embodiment, R includes alkyl substituted with hydroxy.

The term “aryl” includes aromatic hydrocarbyl, including fused aromatic rings, such as, for example, phenyl and naphthyl.

The term “heteroaryl” includes heterocyclic aromatic derivatives having at least one heteroatom such as, for example, nitrogen, oxygen, phosphorus, or sulfur, and includes, for example, furyl, pyrrolyl, thienyl, oxazolyl, pyridyl, imidazolyl, thiazolyl, isoxazolyl, pyrazolyl, isothiazolyl, etc. The term “heteroaryl” also includes fused rings in which at least one ring is aromatic, such as, for example, indolyl, purinyl, benzofuryl, etc.

In some examples, aryl and heteroaryl groups can be unsubstituted or substituted on the ring by, for example, aryl, heteroaryl, alkyl, alkenyl, alkoxy, amino, carboxy, halo, nitro, cyano, —SO₃H, phosphono, or hydroxy. When aryl, aralkyl, or heteroaryl is substituted, preferably the substitution is C₁-C₄ alkyl, halo, nitro, amido, hydroxy, carboxy, sulpho, or phosphono. In one embodiment, R includes aryl substituted with C₁-C₄ alkyl.

As used herein, the term “saturated” refers to a group where all available valence bonds of the backbone atoms are attached to other atoms Representative examples of saturated groups include, but are not limited to, butyl, cyclohexyl, piperidine, and the like.

As used herein, the term “unsaturated” refers to a group where at least one valence bond of two adjacent backbone atoms is not attached to other atoms. Representative examples include, but are not limited to, alkenes (e.g., —CH₂—CH₂CH═CH), phenyl, pyrrole, and the like.

As used herein, the term “substituted” refers to a group having one or more hydrogens or other atoms removed from a carbon or suitable heteroatom and replaced with a further group (i.e., substituent).

As used herein, the term “unsubstituted” refers to a group that does not have any further groups attached thereto or substituted therefore.

In some examples, the peroxycarboxylic acid is a C₁ C₂₂ peroxycarboxylic acid. In some examples, the C₁-C₂₂ peroxycarboxylic acid is a C₂-C₂₀ peroxycarboxylic acid. In some examples, the C₁-C₂₂ peroxycarboxylic acid is a C₁; C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ peroxycarboxylic acid. In some examples, the peroxycarboxylic acid is a C₂-C₁₂ peroxycarboxylic acid, a C₂-C₁₀ peroxycarboxylic acid, a C₂-C₈ peroxycarboxylic acid, a C₂-C₆ peroxycarboxylic acid, a C₂-C₅ peroxycarboxylic acid, a C₂-C₄ peroxycarboxylic acid, a C₂-C₃ peroxycarboxylic acid, or a C₂ peroxycarboxylic acid. In still some examples, the peroxycarboxylic acid comprises peracetic acid.

In terms of acidity, peroxycarboxylic acids are about 1000 times weaker than the parent carboxylic acid, due to the absence of resonance stabilization of the anion. Using peracetic acid as the example, peracetic acid is a weaker acid than the parent acetic acid, with a pKa of 8.2 (c.f. a pKa of 4.76 for acetic acid).

The sporulating composition comprises a first concentration of the peroxycarboxylic acid. The first concentration of the peroxycarboxylic acid is sufficient to cause sporulation of the oocysts within a time period. In some examples, the sporulating composition comprises sufficient peroxycarboxylic acid to cause at least 80% sporulation of the oocysts. As used herein, a reference to “at least 80%” refers to 80%, 81%, 82%, 83%, 84%, 85%, 86%, 87%, 88%, 89%, 90%, 91%, 92%, 93%, 94%, 95%, 96%, 97%, 98%, 99%, 99.5% and 100%. In some examples, the sporulating composition comprises sufficient peroxycarboxylic acid to cause at least 80% sporulation of the oocysts within 72 hours, within 48 hours, or within 24 hours. In some examples, the first concentration comprises less than 20,000 ppm peroxycarboxylic acid, less than 15,000 ppm peroxycarboxylic acid, less than 10,000 ppm peroxycarboxylic acid, less than 9,000 ppm peroxycarboxylic acid, less than 8,000 ppm peroxycarboxylic acid, less than 7,200 ppm peroxycarboxylic acid, less than 7,000 ppm peroxycarboxylic acid, less than 6,000 ppm peroxycarboxylic acid, less than 5,000 ppm peroxycarboxylic acid, less than 4,000 ppm peroxycarboxylic acid, less than 3,000 ppm peroxycarboxylic acid, less than 2,000 ppm peroxycarboxylic acid, less than 1500 ppm peroxycarboxylic acid, less than 1000 ppm peroxycarboxylic acid, less than 900 ppm peroxycarboxylic acid, less than 800 ppm peroxycarboxylic acid, less than 700 ppm peroxycarboxylic acid, less than 600 ppm peroxycarboxylic acid, less than 500 ppm peroxycarboxylic acid, less than 400 ppm peroxycarboxylic acid, less than 300 ppm peroxycarboxylic acid, less than 200 ppm peroxycarboxylic acid, or less than 100 ppm peroxycarboxylic acid. In some examples, the first concentration comprises less than 20,000 ppm peroxycarboxylic acid. In some examples, the first concentration comprises less than 7,200 ppm peroxycarboxylic acid. In some examples, the first concentration comprises less than 2,000 ppm peroxycarboxylic acid. In some examples, the first concentration comprises less than about 1,000 ppm peroxycarboxylic acid. In some examples, the first concentration comprises greater than 1 ppm peroxycarboxylic acid, greater than 5 ppm peroxycarboxylic acid, greater than 10 ppm peroxycarboxylic acid, greater than 50 ppm peroxycarboxylic acid, greater than 100 ppm peroxycarboxylic acid, greater than 150 ppm peroxycarboxylic acid, greater than 200 ppm peroxycarboxylic acid or greater than 250 ppm peroxycarboxylic acid.

The peroxycarboxylic acid may be in a range provided by any two or more of the upper and/or lower amounts, for example between about 10 ppm and 20,000, between about 100 ppm and 5000 ppm, or between about 200 ppm and 2000 ppm. In some examples, the first concentration comprises between about 1,500 ppm and 10 ppm peroxycarboxylic acid. In some examples, the first concentration comprises between about 1,000 ppm and 50 ppm, about 500 ppm and 10 ppm or between about 300 ppm and 10 ppm peroxycarboxylic acid. In some examples, the first concentration comprises about 1,000 ppm peroxycarboxylic acid. In some examples, the first concentration comprises between about 200 to 350 ppm peroxycarboxylic acid. In some examples, the first concentration comprises between about 250 ppm and 300 ppm peroxycarboxylic acid. In some examples, the first concentration comprises about 250 ppm, 260 ppm, 270 ppm, 280 ppm, 290 ppm or 300 ppm of peroxycarboxylic acid. In some examples, the first concentration comprises about 280 ppm of peroxycarboxylic acid. In some examples, the first concentration comprises about 10 to 100 ppm peroxycarboxylic acid. In some examples, the first concentration comprises about 40 to 60 ppm peroxycarboxylic acid.

In some examples, the sporulating composition further comprises hydrogen peroxide. In some examples, the sporulating composition comprises less than 100,000 ppm hydrogen peroxide, less than 75,000 ppm hydrogen peroxide, less than 50,000 ppm hydrogen peroxide, less than 45,000 ppm hydrogen peroxide, less than 40,000 ppm hydrogen peroxide, less than 36,000 ppm hydrogen peroxide, less than 35,000 ppm hydrogen peroxide, less than 30,000 ppm hydrogen peroxide, less than 25,000 ppm hydrogen peroxide, less than 20,000 ppm hydrogen peroxide, less than 15,000 ppm hydrogen peroxide, less than 10,000 ppm hydrogen peroxide, less than 6,000 ppm hydrogen peroxide, less than 4,000 ppm hydrogen peroxide, less than 3,600 ppm hydrogen peroxide, less than 3,200 ppm hydrogen peroxide, less than 2,800 ppm hydrogen peroxide, less than 2,400 ppm hydrogen peroxide, less than 2,000 ppm hydrogen peroxide, less than 1,700 ppm hydrogen peroxide, less than 1,600 ppm hydrogen peroxide, less than 1,200 ppm hydrogen peroxide, less than 1,000 ppm hydrogen peroxide or less than 500 ppm hydrogen peroxide. In some examples, the sporulating composition comprises less than 100,000 ppm hydrogen peroxide. In some examples, the sporulating composition comprises less than 36,000 ppm hydrogen peroxide. In some examples, the sporulating composition comprises less than 10,000 ppm hydrogen peroxide. In some examples, the sporulating composition comprises less than about 5,000 ppm hydrogen peroxide. In some examples, the sporulating composition comprises about 5,000 ppm hydrogen peroxide. In some examples, the sporulating composition comprises about 3,000 ppm hydrogen peroxide. In some examples, the sporulating composition comprises about 2500 ppm hydrogen peroxide. In some examples, the sporulating composition comprises about 2,000 ppm hydrogen peroxide. In some examples, the sporulating composition comprises about 1,700 ppm hydrogen peroxide. In some examples, the sporulating composition comprises about 1250 ppm hydrogen peroxide. In some examples, the sporulating composition comprises about 250 ppm hydrogen peroxide. In some examples, the first concentration comprises greater than 1 ppm hydrogen peroxide, greater than 5 ppm hydrogen peroxide, greater than 10 ppm hydrogen peroxide, greater than 50 ppm hydrogen peroxide, greater than 100 ppm hydrogen peroxide, greater than 150 ppm hydrogen peroxide, greater than 200 ppm hydrogen peroxide or greater than 250 ppm hydrogen peroxide.

The hydrogen peroxide may be in a range provided by any two or more of the upper and/or lower amounts, for example between about 10 ppm and 100,000, between about 50 ppm and 10,000 ppm, or between about 100 ppm and 7,500 ppm. In some examples, the sporulating composition comprises between about 7,500 ppm and 50 ppm hydrogen peroxide. In some examples, the sporulating composition comprises between about 5,000 ppm and 250 ppm hydrogen peroxide. In some examples, the sporulating composition comprises between about 2,000 ppm and 250 ppm hydrogen peroxide.

Any suitable weight ratio of peroxycarboxylic acid to hydrogen peroxide may be used. In some examples, the weight ratio of peroxycarboxylic acid to hydrogen peroxide is from about 1:1 to about 1:10. In some examples, the weight ratio of peroxycarboxylic acid to hydrogen peroxide is from about 1:1 to about 1:6. In some examples, the weight ratio of peroxycarboxylic acid to hydrogen peroxide is from about 1:1 to about 1:5. In some examples, the weight ratio of peroxycarboxylic acid to hydrogen peroxide is about 1:5.

In some examples, the sporulating composition further comprises a carboxylic acid. A carboxylic acid includes any compound of the formula R—(COOH) n in which R can be hydrogen, alkyl, alkenyl, alkyne, acylic, alicyclic group, aryl, heteroaryl, or heterocylic group, and n is 1, 2, or 3. Preferably R includes hydrogen, alkyl, or alkenyl. Any suitable C₁ C₂₂ carboxylic acid can be used in the present compositions. In some examples, the C₁-C₂₂ carboxylic acid is a C₂-C₂₀ carboxylic acid. In some examples, the C₁-C₂₂ carboxylic acid is a C₁; C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ carboxylic acid. In some examples, the carboxylic acid is a C₂-C₁₂ carboxylic acid, a C₂-C₁₀ carboxylic acid, a C₂-C₈ carboxylic acid, a C₂-C₆ carboxylic acid, a C₂-C₅ carboxylic acid, a C₂-C₄ carboxylic acid, a C₂-C₃ carboxylic acid or a C₂ carboxylic acid. In still some examples, the carboxylic acid comprises acetic acid. In some examples, the carboxylic acid is the corresponding acid of the peroxycarboxylic acid. For example, if the peroxycarboxylic acid is peracetic acid, the carboxylic acid is acetic acid.

In some examples, the sporulating composition comprises less than 30,000 ppm carboxylic acid, less than 22,500 ppm carboxylic acid, less than 15,000 ppm carboxylic acid, less than 13,500 ppm carboxylic acid, less than 12,000 ppm carboxylic acid, less than 10,800 ppm carboxylic acid, less than 10,500 ppm carboxylic acid, less than 9,000 ppm carboxylic acid, less than 7,500 ppm carboxylic acid, less than 6,000 ppm carboxylic acid, less than 4,500 ppm carboxylic acid, less than 3,000 ppm carboxylic acid, less than 2,000 ppm carboxylic acid, less than 1,000 ppm carboxylic acid, less than 900 ppm carboxylic acid, less than 800 ppm carboxylic acid, less than 700 ppm carboxylic acid, less than 600 ppm carboxylic acid, or less than 500 ppm carboxylic acid. In some examples, the sporulating composition comprises less than 30,000 ppm carboxylic acid. In some examples, the sporulating composition comprises less than 10,800 ppm carboxylic acid. In some examples, the sporulating composition comprises less than 3,000 ppm carboxylic acid. In some examples, the sporulating composition comprises less than about 1,500 ppm carboxylic acid. In some examples, the sporulating composition comprises less than about 1,000 ppm carboxylic acid. In some examples, the sporulating composition comprises less than about 500 ppm carboxylic acid.

In some examples, the first concentration comprises greater than 1 ppm carboxylic acid, greater than 5 ppm carboxylic acid, greater than 10 ppm carboxylic acid, greater than 50 ppm carboxylic acid, greater than 100 ppm carboxylic acid, greater than 150 ppm carboxylic acid, greater than 200 ppm carboxylic acid or greater than 250 ppm carboxylic acid.

The carboxylic acid may be in a range provided by any two or more of the upper and/or lower amounts, for example between about 10 ppm and 30,000, between about 50 ppm and 10,000 ppm, or between about 100 ppm and 7,500 ppm. In some examples, the sporulating composition comprises between about 2,250 ppm and 15 ppm carboxylic acid. In some examples, the sporulating composition comprises between about 1,500 ppm and 75 ppm carboxylic acid, for example between about 1,000 ppm and 300 ppm. In some examples, the sporulating composition comprises about 750 ppm carboxylic acid. In some examples, the sporulating composition comprises about 416 ppm carboxylic acid. In some examples, the sporulating composition comprises about 375 ppm carboxylic acid. In some examples, the sporulating composition comprises between about 350 and about 450 ppm carboxylic acid. In some examples, the sporulating composition comprises about 75 ppm carboxylic acid.

Commercially available peroxycarboxylic acids are typically sold as an equilibrium solution. These equilibrium solutions usually contain the peroxycarboxylic acid as well as the corresponding carboxylic acid, hydrogen peroxide and water. Accordingly, in some examples the sporulation media further comprises hydrogen peroxide and/or the corresponding carboxylic acid. In some examples, the sporulation media comprises a C₁-C₂₂ carboxylic acid; a C₁-C₂₂ peroxycarboxylic acid; and hydrogen peroxide.

In some examples, the peroxycarboxylic acid is peracetic acid. Peracetic acid (also known as peroxyacetic acid, or PAA) is an organic compound with the formula CH₃CO₃H. It is a colourless, water soluble liquid which can be used as a biocide for various microorganisms, such as bacteria, virus, yeast, fungi and spores. For example, peracetic acid is used sterilise medical, surgical, and dental instruments chemically (e.g., endoscopes, arthroscopes). As would be understood by the person skilled in the art, the corresponding carboxylic acid of peracetic acid is acetic acid. Accordingly, in some examples, the sporulation composition comprises peracetic acid; acetic acid; and hydrogen peroxide. In some examples, the sporulating composition comprises peracetic acid, hydrogen peroxide, carboxylic acid and a solvent, e.g., water. In some examples, the sporulating composition comprises or consists of peracetic acid, hydrogen peroxide, carboxylic acid, water and optionally one or more additives, e.g. an antifoam or anti-aggregation agent. In some examples, the sporulation composition comprises peracetic acid; acetic acid; and hydrogen peroxide at a weight ratio of 3-7:18-30:6-10. In some examples, the sporulation composition comprises peracetic acid; acetic acid; and hydrogen peroxide at a weight ratio of 4-6:25-29:7-8. In some examples, the sporulation composition comprises peracetic acid; acetic acid; and hydrogen peroxide at a weight ratio of 4.8-6:18-26:7-8. In some examples, the sporulation composition comprises peracetic acid; acetic acid; and hydrogen peroxide at a weight ratio of 5:25:7.5.

As would be understood by the person skilled in the art, peroxycarboxylic acids, such as peracetic acid, are not prepared by combining hydrogen peroxide and the corresponding carboxylic acid in water. A catalyst is required to produce the peroxycarboxylic acid. In some examples, the catalyst is an acidic catalyst (e.g. a homogeneous acidic catalyst) in accordance with the reaction scheme below:

H₂O₂+CH₃CO₂H═H₂O+CH₃CO₃H

Suitable acidic catalysts include an inorganic acid or solid acid catalyst. In some examples, a solid acid catalyst is an acidic cation-exchange resin or a perfluorinated sulfonic resin or immobilized sulfuric acid. In some examples, the inorganic acid is phosphoric acid or sulfuric acid. In some examples, the inorganic acid is sulfuric acid.

The methods described herein comprise incubating viable oocysts with a sporulating composition comprising a first concentration of peroxycarboxylic acid to produce viable, sporulated Eimeria oocysts. As used herein, the amount of oocysts as well as any other insoluble matter remaining after isolation of the oocysts from faecal matter added to the sporulating composition is referred to as the solids content. The solids content is typically expressed as a percentage and, in one example, is calculated by measuring wet pellet volume after centrifugation divided by the total suspension volume. For example, 5 ml wet pellet after centrifugation of 100 mL material represents 5% solids. Solids include, but are not limited to, viable and unviable Eimeria oocysts, cellular debris, dead bacteria, plant feed residue and any material that co-isolates during oocyst separation. In some examples, the solids content is 10% v/v or less, 9% or less, 8% or less, 7% or less, 6% or less, 5% or less, 4% or less, 3% or less, 2% or less or 1% or less. In some examples, the solids content is between about 0.1 and 10%, 0.1 and 9%, 0.1 and 8%, 0.1 and 7%, 0.1 and 6% or 0.1 and 5%. In some examples, the solids content is about 5%. In some examples, the solids content is about 2%. In some examples, the oocysts are incubated with the sterilising composition at a solids content of between about 0.05% v/v and 10% v/v, or between about 1% v/v and 6% v/v, or between about 4.5% v/v and 5.5% v/v. In some examples, the ratio of sporulation medium to solids is about 1:0.01, about 1:0.025, about 1:0.05, or about 1:0.1 (v/v).

Solid content can be also measured indirectly by infrared turbidimetry, and solid load correlated to Nephelometric Turbidity Units (NTU). In some examples, turbidity is measured using HI98703 (Hanna) or IC-TN400 (Apera). The IC-TN400 is preferred as it is not biased by sample colour. In some examples, the turbidity of the incubated composition (e.g. after addition of the oocysts to the sporulation composition) is 7000 NTU or less, 6000 NTU or less, 5000 NTU or less, 4200 NTU or less, 3500 NTU or less, 2800 NTU or less, 2100 NUT or less, 1400 NTU or less, 700 NTU or less, 350 NTU or less, 280 NTU or less, 210 NTU or less, 140 NTU or less or 70 NTU or less. In some examples, the turbidity is between about 5000 NTU and about 70 NTU, or about 3500 NTU and about 700 NTU. In some examples, the turbidity is about 3500 NTU. In some examples, the turbidity is about 1400 NTU. In some examples, the turbidity is about 700 NTU. As demonstrated in the examples hereinbelow, 700 NTU corresponds to 1% solids load.

Described herein is a method of sporulating oocysts (e.g., Eimeria oocysts), comprising the steps of providing a composition comprising oocysts, and sporulating the oocysts in a sporulation medium as described herein for a time and under conditions suitable for sporulation. In some examples, there is provided a method for sporulating viable Eimeria oocysts comprising: incubating the oocysts with a sporulating composition comprising a less than 20,000 ppm peroxycarboxylic acid (e.g. 2% Proxitane) to produce viable, sporulated Eimeria oocysts. The method optionally comprises collecting the oocysts from an infected animal (e.g. the faeces or caeca of an infected animal), separating the oocysts from extraneous material. The oocysts may then be transferred to the sporulation vessel, and sporulation allowed to proceed for a time and under conditions suitable for sporulation. In some examples, the sporulating composition may be stirred or agitated and aerated during the sporulation process.

The present inventors have found that use of a peroxycarboxylic acid (e.g. peracetic acid) for sporulating oocysts may be advantageous in that it may reduce the bioburden from the start of the sporulation step, provide high sporulation rates, enable single vessel sporulation and sterilisation and/or may be more easily disposed of than conventional sporulation media containing potassium dichromate.

In some examples, the sporulation composition further comprises a compound which reduces aggregation of the oocysts and/or sticking of debris to the oocysts. Any suitable compound may be used. These include detergents, protein, peptides, protein hydrolysate and/or amino acids. For example, soy protein, soy hydrolysate, casein, casein hydrolysate, lysozyme, albumen, bovine serum albumen, milk proteins, amino acids (e.g., arginine, phenylalanine and/or aspartic acid), fetal calf serum, chicken serum, whole milk, and the like may be used to reduce aggregation and/or sticking. In some examples, the compound is a detergent. In some examples, the detergent is an anionic detergent. Suitable anionic detergents include, but are not limited to, dioctyl sulfosuccinate and salts thereof (also referred to as docusate). Salts of docusate include sodium docusate, calcium docusate and potassium docusate. In some examples, the salt is sodium docusate. In some examples, the compound which reduces aggregation of the oocysts is an anti-foaming agent, such as Antifoam A. Typically, the concentration of compound which reduces aggregation of the oocysts will be about 0.05 w/v % to 1 w/v %, about 0.1 w/v % to 0.5 w/v % or about 0.25 w/v %. In some examples, the sporulation composition comprises docusate (e.g. sodium docusate) in an amount of 0.25 w/v %. In some examples, a detergent (such as docusate) is not added.

The sporulating step may be carried out for any suitable length of time. In some examples, the sporulating step is carried out for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 36 hours, at least 48 hours, at least 60 hour or at least 72 hours. In some examples, the sporulating step is carried out for between 12 hours and 48 hours, between 18 hours and 48 hours, between about 24 hours and 48 hours, between about 30 hours and 48 hours, between about 36 hours and 48 hours or between 42 hours and 48 hours. In some examples, the sporulating step is carried out for about 36 to 48 hours.

The temperature at which the sporulating step is performed should be suitable for sporulation and to maintain the viability of the population of oocysts. Preferably, the oocysts are not subjected to freezing or prolonged exposure to high temperatures. In some examples, the sporulating step is performed at a temperature of between about 20° C. and about 37° C. In some examples, the sporulating step is performed at a temperature of between about 22° C. and about 32° C. In some examples, the sporulating step is performed at a temperature of between about 25° C. and about 30° C. In some examples, the sporulating step is performed at a temperature of about 25° C., 26° C., 27° C., 28° C., 29° C., or 30° C. In some examples, the sporulating step is performed at a temperature of about 27° C. In some examples, the sporulating step is performed at a temperature of between about 25° C. and 30° C., for about 12 to 36 hours. In some examples, the sporulating step is performed at a temperature of about 27° C. and 30° C., for about 24 hours.

In some examples, the pH of the sporulating composition is less than about 7, for example less than about 6, less than about 5, less than about 4, less than about 3.5, or less than about 3. In some examples, the pH of the sporulating composition is between about 2 and about 7, between about 2 and about 6, between about 2 and about 5 or between about 2 and about 4. In some examples, the pH of the sporulating composition is between about 2.5 and about 4.

In some examples, the sporulation step is performed in a reaction vessel having a headspace of at least 50%, at least 60%, at least 70%, at least 80% or at least 90%. In some examples, the sporulation step is performed in a reaction vessel having a headspace of about 80%.

### Bioburden Reduction and Sterilisation

Generally, it is advantageous to reduce the bioburden of the oocysts after sporulation as subsequent steps will be carried out using sterile procedures and reagents. Traditionally, oocysts for use in vaccines are treated with sodium hypochlorite followed by formaldehyde for bioburden reduction and sterilisation. This is thought to cause damage to the oocyst wall, weakening the structure of the oocyst and potentially reducing the long-term viability of the sanitized oocysts during storage. It is also thought to cause clumping of the oocysts which can lead to damage and make cell counting more challenging. The use of different chemicals also increases manual handling and requires a large number of centrifugation, resuspension and transfer steps. The present inventors have found that peroxycarboxylic acids as described herein are also suitable for the sterilisation of oocyst preparations. Use of a peroxycarboxylic acid for both sporulation and sterilisation (including bioburden reduction) reduces manually handling and the means that process can be completed in a single reaction vessel. Accordingly, the present application also provides a method of sterilising a composition of viable oocysts, comprising the step of providing a composition containing oocysts, and sanitizing the oocysts in a sterilising composition for a time and under conditions sufficient to achieve the desired level of sterilisation of the composition.

Following sporulation (e.g. as described herein), the oocyst preparation may be subjected to optional purification procedures (e.g., density filtration, flotation, buffer exchange and the like), followed by sterilisation. In preferred examples, the sterilisation step follows the sporulation step without any intervening purification procedures. In some examples, the sterilisation step immediately follows the sporulation step. In some examples, the sporulation step and the sterilisation step are performed in a single reaction vessel.

As used herein, “sterilising” or “sterilisation” or “sterilised” refers to a reduction in the contaminating (i.e. not Eimeria) microbial load (e.g., viable contaminating microorganisms, as defined herein) in the oocyst preparation. Contaminating microorganisms include, but are not limited to, bacteria, mold, fungi, yeast and/or viruses. It is not necessary that the oocyst preparation contain absolutely no microbial contamination, as long as the final preparation is suitable for its intended use (e.g., as a vaccine). In the case of vaccines (e.g. vaccines intended for administration to birds), the preparation will generally be essentially free of detectable microbial contamination (i.e., no significant levels of contaminating microorganisms are detected). In some examples, there is at least about a 50%, 60%, 75%, 85%, 90%, 95%, 99% or more reduction in detectable contaminating microorganisms as compared with the level in the absence of sanitization.

Methods of detecting microorganisms are known in the art and depend on the class of microorganism being detected. In some examples, microorganisms are detected by direct inoculation of the oocysts preparation into a suitable growth media (e.g. Tryptic Soy Broth (TSB)) or onto suitable plates and the inoculated media/plates incubated under suitable conditions for microorganism growth. Microorganism growth may be monitored using techniques known to the person skilled in the art, for example, visual inspection and/or OD₆₀₀ measurements.

In some examples, there is provided a method for preparing a sterilised formulation comprising viable Eimeria oocysts, the method comprising: incubating the viable, sporulated Eimeria oocysts with a sterilising composition comprising a second concentration of the peroxycarboxylic acid relative to the sporulating composition to produce the sterilised formulation. In some examples, there is provided a method for preparing a formulation comprising viable Eimeria oocysts, the method comprising: incubating viable oocysts with a sporulating composition comprising a first concentration of peroxycarboxylic acid to produce viable, sporulated Eimeria oocysts; and incubating the viable, sporulated Eimeria oocysts with a sterilising composition comprising a second concentration of the peroxycarboxylic acid to produce the formulation, wherein the second concentration of peroxycarboxylic is greater than the first concentration. In some examples, the second concentration of peroxycarboxylic is increased relative to the first concentration of peroxycarboxylic acid.

The second concentration of peroxycarboxylic acid is greater than the first concentration. In some examples, the second concentration of peroxycarboxylic acid is greater than 250 ppm. In some examples, the second concentration of peroxycarboxylic acid is at least about 300 ppm, 350 ppm, 400 ppm, 450 ppm, 500 ppm, 550 ppm, 600 ppm, 650 ppm, 700 ppm, 750 ppm, 800 ppm, 850 ppm, 900 ppm, 950 ppm, or 1000 ppm. In some examples, the second concentration of peroxycarboxylic acid is at least about 500 ppm. In some examples, the second concentration of peroxycarboxylic acid is at least about 900 ppm. In some examples, the second concentration of peroxycarboxylic acid is at least about 1000 ppm. In some examples, the second concentration of peroxycarboxylic acid is at least about 1100 ppm. In some examples, the second concentration of peroxycarboxylic acid is between about 500 ppm and 2000 ppm, between about 1000 ppm and 1500 ppm, or between about 1000 ppm and 1250 ppm. In some examples, the second concentration of peroxycarboxylic acid is about 1100 ppm. In some examples, the second concentration of peroxycarboxylic acid is about 1200 ppm.

In some examples, the first concentration of peroxycarboxylic acid is 300 ppm or less and the second concentration of peroxycarboxylic acid is greater than 300 ppm. In some examples, the first concentration of peroxycarboxylic acid is between about 10 and 300 ppm and the second concentration of peroxycarboxylic acid is between about 800 and 1300 ppm. In some examples, the first concentration of peroxycarboxylic acid is between about 40 and 300 ppm and the second concentration of peroxycarboxylic acid is between about 800 and 1300 ppm. In some examples, the first concentration of peroxycarboxylic acid is about 40-50 ppm and the second concentration of peroxycarboxylic acid is about 1000-1200 ppm. In some examples, the first concentration of peroxycarboxylic acid is about 250-300 ppm and the second concentration of peroxycarboxylic acid is about 1000-1200 ppm.

As would be appreciated by the person skilled in the art, the peroxycarboxylic acid added for sporulation and/or sterilisation will be consumed during the sporulation and/or sterilisation steps. Accordingly, the first concentration of peroxycarboxylic acid refers to the amount of peroxycarboxylic acid added at the initiation of the sporulation step. The second concentration of peroxycarboxylic acid refers to the amount of additional peroxycarboxylic acid added at the initiation of the sterilisation step and not the total concentration of peroxycarboxylic acid added to the composition over the two method steps. For the avoidance of doubt, the total concentration of peroxycarboxylkic acid added while performing the method is the sum of the first and second concentrations. For example, when the first concentration of peroxycarboxylic acid is about 250 ppm and the second concentration of peroxycarboxylic acid is about 1000 ppm, the total concentration of peroxycarboxylic acid added to the composition over the course of the method is about 1250 ppm.

Typically, the sterilisation process results in a composition comprising viable oocysts having a level of microbial contamination that is suitable for administration to an animal subject (e.g., as a vaccine).

The sterilising step may be carried out for any suitable length of time. In some examples, the sterilising step is carried out for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 36 hours or at least 48 hours. In some examples, the sterilising step is carried out for between 12 hours and 36 hours, between 18 hours and 30 hours or between 20 hours and 28 hours. In some examples, the sterilising step is carried out for about 24 hours.

The temperature at which the sporulating step is performed should be suitable for sterilisation and to maintain viability the population of oocysts. Preferably, the oocysts are not subjected to freezing or prolonged exposure to high temperatures. In some examples, the sterilisation step is performed at a temperature of between about 20° C. and about 37° C. In some examples, the sterilisation step is performed at a temperature of between about 22° C. and about 32° C. In some examples, the sterilisation step is performed at a temperature of between about 25° C. and about 30° C. In some examples, the sterilisation step is performed at a temperature of about 25° C., 26° C., 27° C., 28° C., 29° C., or 30° C. In some examples, the sterilisation step is performed at a temperature of about 27° C. In some examples, the sterilisation step is performed at a temperature of between about 25° C. and 30° C., for about 12 to 36 hours. In some examples, the sterilisation step is performed at a temperature of about 27° C. and 30° C., for about 24 hours.

The ratio of solids to sanitization medium (referred to as the solids content) is not critical as long as it is sufficient to reduce microbial growth to the desired level. In some examples, the ratio of sterilisation medium to solids is about 1:0.01, about 1:0.025, about 1:0.05, or about 1:0.1 (v/v). In some examples, the solids content is about 1%, about 0.5%, about 1%, about 2.5% or about 5%.

In some examples, the turbidity of the incubated composition is 7000 NTU or less, 6000 NTU or less, 5000 NTU or less, 4200 NTU or less, 3500 NTU or less, 2800 NTU or less, 2100 NUT or less, 1400 NTU or less, 700 NTU or less, 350 NTU or less, 280 NTU or less, 210 NTU or less, 140 NTU or less or 70 NTU or less. In some examples, the turbidity is between about 5000 NTU and about 70 NTU, or about 3500 NTU and about 700 NTU. In some examples, the turbidity is about 3500 NTU. In some examples, the turbidity is about 1400 NTU. In some examples, the turbidity is about 700 NTU.

Advantageously, the methods described herein may be carried out in a single vessel (e.g. single reaction vessel), for example the incubating may be performed in a single vessel. Any suitable vessel may be used. Traditionally methods for the preparation of oocysts for vaccines have used potassium dichromate for the sporulation step and a sodium hypochlorite solution for sterilisation. This requires purification and/or isolation of oocysts between the sporulation and sterilisation steps. Advantageously, the methods described herein use the same chemical for the sporulation and sterilisation step. The present inventors have found that increasing the concentration of the peroxycarboxylic acid in the sterilisation media relative to the sporulation media helps ensure that the oocyst preparation is suitable for use in vaccines. It is also thought to help reduce any potential damage caused by incubating unsporulated oocysts with higher concentrations of peroxycarboxylic acid. In carrying out sterilisation after the sporulation period, the concentration of peroxycarboxylic acid is increased by the addition of a second concentration of peroxycarboxylic acid or a composition comprising peroxycarboxylic acid (for example, Proxitane). This can be achieved by adding the additional peroxycarboxylic acid to composition after a suitable period of time, for example after the end of the sporulation step or time assigned for the sporulation step. As would be understand sporulation of oocysts is not necessarily complete at the time the peroxycarboxylic acid concentration is increased to the second concentration and sporulation may continue for some or all of the sterilisation step. The oocysts are then incubated for a time and under conditions suitable for sterilisation. In some examples, the solution may be stirred or agitated and aerated during the sterilisation process. For the avoidance of doubt, the first concentration is added to the reaction vessel with the oocysts at the initiation of the sporulation step and the second concentration is added to the reaction vessel at the initiation of the sterilisation step. The total concentration of peroxycarboxylic acid added to the reaction vessel is the sum of the first and the second concentrations of peroxycarboxylic acid.

In some examples, the sterilisation composition further comprises a compound which reduces aggregation of the oocysts. Any suitable compound may be used as described herein. In some examples, compound which reduces aggregation of the oocysts is a detergent, e.g. an anionic detergent such as docusate or a salt thereof.

In some examples, a detergent is not added.

In some examples, the pH of the sterilising composition is less than about 7, for example less than about 6, less than about 5, less than about 4, less than about 3.5, or less than about 3. In some examples, the pH of the sterilising composition is between about 2 and about 7, between about 2 and about 6, between about 2 and about 5 or between about 2 and about 4. In some examples, the pH of the sterilising composition is between about 2.5 and about 4.

In some examples, the sterilisation step is performed in a reaction vessel having a headspace of at least 50%, at least 60%, at least 70%, at least 80% or at least 90%. In some examples, the sporulation step is performed in a reaction vessel having a headspace of about 80%.

### Purification

Following sporulation and/or sterilisation (e.g. as described herein), the oocyst preparation may be subjected to purification procedures (e.g., buffer exchange, concentration, and the like). In some examples, this step is suitable to remove or reduce the amount of the peroxycarboxylic acid, as well as other unwanted components of the sporulation and/or sterilisation composition. In some examples, this step is suitable to remove or reduce the amount of the peroxycarboxylic acid, hydrogen peroxide and/or carboxylic acid. In some examples, this step is suitable to remove or reduce the amount of the peracetic acid. In some examples, this step is suitable to remove or reduce the amount of Proxitane. In some examples, this step is suitable to exchange the sporulation and/or sterilisation media for a buffer composition. In some examples, the sporulation step is followed by one or more purification procedures. In some examples, the sterilisation step is followed by one or more purification procedures. Generally, when the one or more purification procedures are carried out after the sterilisation step they will be carried out using sterile procedures and reagents.

In some examples, the sterilised formulation is processed to substantially remove the peroxycarboxylic acid (e.g. the peroxycarboxylic acid added during sporulation and/or sterilisation). Any suitable method of removing peroxycarboxylic acid may be used. In some example, the peroxycarboxylic acid is substantially removed by buffer exchange. Suitable techniques include, but are not limited to dialysis, centrifugation and resuspension, and/or diafiltration and the like. In some examples, processing comprises diafiltration. In some examples, processing comprises diafiltration, e.g. tangential flow filtration (TFF). In some examples, processing comprises diafiltering the sterilised formulation by one or more steps of TFF. In some examples, the at least one step of TFF may comprise at least one diafiltration step using TFF and/or at least one concentration step using TFF. The diafiltration and concentration step may be performed separately but they may also at least partially overlap. In some examples, the at least one step of TFF may comprise at least one diafiltration step using TFF. The at least one or more steps of TFF may efficiently remove the components of the sterilisation buffer, such as peroxycarboxylic acid, hydrogen peroxide, carboxylic acid, and other components such as salts and detergents. The use of TFF may thus reduce the number of steps required to remove or replace the sterilisation composition, help avoid compacting and potential clumping of the oocysts, enable use of a single vessel and/or make it easier to maintain a sterile composition. In some examples, the TFF is a continuous process. Any suitable shear rate may be used. In some examples, the shear rate is less than about 2000 s⁻¹, less than about 1500 s⁻¹, or about 1000 s⁻¹. In some examples, the shear rate is between about 2000 s⁻¹ and about 1000 s⁻¹. In some examples, the shear rate is about 1000 s⁻¹.

Membranes with different molecular weight cutoffs (MWCO) may be used for TFF. In the context of the present disclosure microfiltration membranes are preferably used for TFF. As used herein, “microfiltration” refers to filtration through a membrane with pores, of for example, approximately 0.1-10 μm in diameter. Microfiltration membranes are typically defined by the pore size of the membrane of greater than 0.1 μm, for example 0.2 μm.

Two filter configurations are typically used for TFF. In cartridge filters (also called hollow fibre filters), the membrane forms a set of parallel hollow fibres. The feed stream passes through the lumen of the fibres and the permeate is collected from outside the fibres. Cartridges are characterized in terms of fibre length, lumen diameter and number of fibres, as well as filter pore size. In cassette filters, several flat sheets of membrane are held apart from each other and from the cassette housing by support screens. The feed stream passes into the space between two sheets and permeate is collected from the opposite side of the sheets. Cassettes are characterized in terms of flow path length and channel height, as well as membrane pore size. The channel height is determined by the thickness of the support screen. TFF may be carried out using any suitable filter membrane. For example, TFF may be carried out using a TFF hollow fibre membrane (i.e. cartridge filter) or a TFF membrane cassette. In some examples, the use of a TFF hollow fibre membrane cartridge (i.e. cartridge filter) is preferred. The molecular weight cutoff of the filter membrane can be selected by the person skilled in the art such that it is suitable for retaining the Eimeria oocysts in the retentate while allowing unwanted components to pass through the filter. In some examples, the pore size of the filter membrane is about 0.1 μm, about 0.2 μm, about 0.45 μm or about 0.6 μm. In some examples, the pore size of the filter membrane is about 0.2 μm. In some examples, the pore size of the filter membrane is greater than about 0.1 μm, greater than about 0.2 μm, greater than about 0.45 μm or greater than about 0.6 μm.

The filter membrane may comprise any suitable filter material, e.g. polyethersulfone (PES), modified polyethersulfone (mPES), polysulfone (PS), modified polysulfone (mPS), polyvinylidene fluoride (PVDF), modified polyvinylidene fluoride (mPVDF), ceramics, metals, polypropylene (PP), cellulose, regenerated cellulose or a cellulose derivative e.g. cellulose acetate or combinations thereof. In some examples, the filter membrane may comprise polysulfone. A hollow fiber filter can be selected based on lumen number, diameter, and length. The filter membrane may comprise any suitable surface area, for example between 0.12 to 1.15 m² (1.3 to 12.5 ft²). In some examples the surface area is 0.84 m² (9 ft²). In some examples the surface area is 0.12 m² (1.3 ft²). Any suitable flow path length may also be used, for example between 30 to 110 cm. In some examples, the flow pathlength is 30 or 60 cm. Any suitable lumen diameter may also be used, for example between 0.25 to 3 mm. In some examples, the lumen diameter is about 0.25, 0.5, 0.75 or 1 mm. In some examples, the lumen diameter is about 1 mm. Examples of suitable TFF membranes include CFP-2-E-9A and CFP-1-E-5A available from Cytiva. In some examples, the TFF membrane is the CFP-2-E-9A. In some examples, the TFF membrane is the CFP-1-E-5A.

In some examples, buffer exchange is used to exchange the sterilisation composition for a buffer, for example a pharmaceutically acceptable buffer. Pharmaceutically acceptable buffers are as defined herein. In some examples, buffer exchange is used to exchange the sterilisation composition for PBS-T80. In some examples, the buffer further comprises a peroxycarboxylic acid as defined herein. In some examples, the buffer further comprises peracetic acid. In some examples, the peroxycarboxylic acid (e.g. peracetic acid) is added after completion of TFF and optionally during formulation of the final composition (e.g. vaccine composition, pharmaceutical composition, immunogenic composition and the like). In some examples, the concentration of peroxycarboxylic acid (e.g. peracetic acid) is sufficient to function as a preservative. In some examples, the concentration of peroxycarboxylic acid (e.g. peracetic acid) is between about 1 ppm and 1000 ppm, between about 1 ppm and 500 ppm, between about 1 ppm and 200 ppm, or between about 1 ppm and 100 ppm. In some examples, the concentration of peroxycarboxylic acid (e.g. peracetic acid) is between about 10 ppm and about 100 ppm.

### Compositions

The methods described herein can be used to produce a composition comprised Eimeria oocysts. Accordingly, the present disclosure also provides a composition comprising Eimeria oocysts produced by or during the method described herein. In some examples, the is provided a composition comprising viable Eimeria oocysts and peroxycarboxylic acid at a concentration of 1000 ppm or less. In some examples, the is provided a composition comprising viable Eimeria oocysts and peroxycarboxylic acid at a concentration of 500 ppm or less. In some examples, the is provided a composition comprising viable Eimeria oocysts and peroxycarboxylic acid at a concentration of less than 300 ppm. In some examples, the is provided a composition comprising viable Eimeria oocysts and peroxycarboxylic acid at a concentration of about 1000 ppm. In some examples, the composition is a sterilised composition. In some examples, the is provided a composition comprising viable Eimeria oocysts and peroxycarboxylic acid at a concentration of between about 200-300 ppm. In some examples, the at least one peroxycarboxylic acid is as defined herein, for example peracetic acid. In some examples, the solids content of the composition is as define herein for the sporulation and/or sterilisation step. In some examples, the solids content is about 1%, about 2% or about 5% by volume. In some examples, the Eimeria oocysts are selected from the group consisting of E. tenella, E. acervulina, E. maxima, E. necatrix, E. mitis, E. praecox, E. mivati, E. lata, E. nagambie, E. zaria and E. brunetii and combinations thereof. In some examples, the Eimeria oocysts are selected from the group consisting of oocysts from E. maxima, E. mitis, E. tenella, E. acervulina, E. brunetti, E. necatrix, E. praecox and combinations thereof. In some examples, the viable Eimeria oocysts comprise unsporulated oocysts. In some examples, the viable Eimeria oocysts comprise sporulated oocysts.

In some examples, there is provided a composition comprising viable population of sporulated Eimeria oocysts, wherein the composition is sterile and, wherein the sporulated oocysts have been treated with a first concentration of peroxycarboxylic acid and a second concentration of peroxycarboxylic acid in a single vessel, wherein the first concentration of peroxycarboxylic acid is less than the second concentration of peroxycarboxylic. In some examples, there is provided a composition comprising viable population of sporulated Eimeria oocysts, wherein the composition is sterile and, wherein the sporulated oocysts have been treated with a first concentration of peroxycarboxylic acid and a second concentration of peroxycarboxylic acid in a single vessel, wherein the first concentration of peroxycarboxylic acid is less 1000 ppm and the second concentration of peroxycarboxylic increased relative to the first concentration. In some examples, the first concentration of peroxycarboxylic acid is less than 500 ppm and the second concentration of peroxycarboxylic greater than about 800 ppm. In some examples, the first concentration of peroxycarboxylic acid is less than 300 ppm and the second concentration of peroxycarboxylic greater than about 900 ppm. In some examples, the first concentration of peroxycarboxylic acid is between about 200 ppm and 300 ppm and the second concentration of peroxycarboxylic greater between about 800 ppm and 1200 ppm. In some examples, the at least one peroxycarboxylic acid is peracetic acid. In some examples, the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof. In some examples, the composition is further processed to substantially remove the peroxycarboxylic acid, preferably wherein the composition is diafiltered using TFF.

In some examples, the composition is a pharmaceutical composition and/or an immunogenic composition. These compositions can be prepared by any techniques known to the person skilled in the art.

In some examples, there is provided a pharmaceutical composition comprising Eimeria oocysts produced by the method described herein, and a pharmaceutically acceptable carrier. As used herein, “pharmaceutically acceptable” refers to a material that is not biologically or otherwise undesirable, i.e., the material may be administered to a subject without causing any undesirable biological effects. Thus, such a pharmaceutical composition may be used, for example, to prepare compositions for immunization. Physiologically and pharmaceutically acceptable carriers may contain other compounds including but not limited to stabilizers, salts, buffers, adjuvants and/or preservative (e.g., antibacterial, antifungal and antiviral agents) as is known in the art. The pharmaceutically acceptable carrier need not be sterile, although it generally will be for in ovo administration to avian embryos or for administration to the eye (for example, via eye drops).

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991). In some examples, the pharmaceutically acceptable carrier comprises PBS-T80.

The composition may optionally comprise pharmaceutically acceptable excipients. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxy toluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

Formulation of the Eimeria oocysts to be administered will vary according to the route of administration and formulation (e.g., solution, emulsion, capsule) selected. An appropriate pharmaceutical composition comprising the Eimeria oocysts produced using the methods described herein can be prepared in a pharmaceutically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. In some examples, the composition comprises PBS-T80.

Different oocyst preparations (e.g., from different species or strains) may be combined to form the composition described herein, for example, a vaccine against multiple protozoan (e.g., Eimeria) species. In some examples, the composition comprises oocysts from one or more Eimeria species (e.g. pathogenic Eimeria species). The person skilled in the art would be able to select the one or more Eimeria species included in the composition based on Eimeria species know to infect or determined to infect the subject to be treated. For example, when the subject is a chicken, the one or more Eimeria species are selected from E. tenella, E. necatrix, E. acervulina, E. maxima, E. brunetti, E. mitis, and E. praecox. When the subject is a turkey, the one or more Eimeria species are selected from E adenoeides, E dispersa, E gallopavonis, and E meleagrimitis. When the subject is a Pheasant, the one or more Eimeria species are selected from E. phasiani, E colchici, E duodenalis, E tetartooimia, and E pacifica. When the subject is a partridge (e.g. Chukar partridge), the one or more Eimeria species are selected from E kofoidi and E legionensis. When the subject is a quail (e.g. Bobwhite quail), the one or more Eimeria species are selected from E lettyae, E dispersa, and E coloni. Wild-type Eimeria are generally isolated from outbreaks of clinical disease in poultry flocks and may be propagated for use in the methods and compositions described herein. Suitable Eimeria species include wildtype strains, precocious or otherwise selected strains, attenuated strains, and oocysts that have been attenuated, e.g., by irradiation, chemical treatment and the like. Further, the term “Eimeria” also includes any newly-discovered strains or species of Eimeria.

In some examples, the composition is an immunogenic composition (e.g. a vaccine). When the composition is a vaccine composition or an immunogenic composition the carrier may be water, typically pyrogen-free water; isotonic saline or buffered (aqueous) solutions, e.g. phosphate, citrate etc. buffered solutions. In some examples, the composition comprises phosphate buffered saline. In some examples, the composition comprises phosphate buffered saline and Tween-80. In some examples, the composition may also comprise formaldehyde.

Any route of administration of the immunogenic composition known in the art may be employed as long as an active immune response (preferably, a protective immune response) against the protozoa is elicited, for example, spray, intravenous, intramuscular, subcutaneous, oral (including in the feed and/or drinking water), intranasal, intradermal, and/or intraperitoneal vaccination. When the subject is a bird (e.g. poultry), the immunogenic composition may be administered in ovo or post-hatch. Examples of post-hatch administration include administration via the eye (e.g. eye drop), administration via feed or drinking water, administration via spray onto the birds or administration by spray using a gel carrier. The artisan will appreciate that the vaccine composition is preferably formulated appropriately for each type of recipient animal and route of administration.

Immunogenic compositions (e.g., a vaccine) produced using the methods described herein may be administered to an animal subject (e.g. poultry) to vaccinate against a protozoan disease. An immunogenic composition (e.g., a vaccine) containing oocysts produced using the methods described herein may be administered to elicit an immunogenic response. Typically, the immunogenic composition comprises an immunogenic amount of oocysts as disclosed herein in combination with a pharmaceutically-acceptable carrier. An “immunogenic amount” is an amount of the oocysts that is sufficient to initiate or evoke an immune response in the subject to which the immunogenic composition is administered. As understood by those skilled in the art, the immunogenic composition may be formulated with live, attenuated and/or killed organisms. In the case of live vaccine, the amount of oocysts is generally sufficient to yield a relatively low level initial infection, which is then followed by one or more rounds of re-infection, via recycling, ultimately leading to immunity.

As would be appreciated by the person killed in the art, an “immunogenic amount” provides just enough infective oocysts to elicit immunity, but not disease in the naïve host. In some examples, the composition is administered at a dose of at least 10 oocysts, at least 20 oocysts, at least 30 oocysts, at least 40 oocysts, at least 50 oocysts, at least 60 oocysts, at least 70 oocysts, at least 80 oocysts, at least 90 oocysts, at least 100 oocysts, at least 110 oocysts, at least 120 oocysts, at least 130 oocysts, at least 140 oocysts, at least 150 oocysts, at least 200 oocysts, at least 250 oocysts, at least 300 oocysts, at least 350 oocysts, at least 400 oocysts, at least 450 oocysts or at least 500 oocysts per species. In some examples, the composition is administered at a dose of at between 100 and 500 oocysts per species. As would be appreciated by the person skilled in the dose of each oocyst species present in the composition may be different. For example, a dose may comprise a minimum of E. acervulina 50 oocysts, E. maxima 100 oocysts, E. mitis 100 oocysts and E. tenella 150 oocysts. In another example, a dose comprises a minimum of E. necatrix 100 oocysts and E. brunetti 50 oocysts. In yet another example, a dose comprises a minimum of E. acervulina 50 oocysts, E. maxima 100 oocysts and E. tenella 150 oocysts. In yet another example, a dose comprises a minimum of E. acervulina 50 oocysts, E. maxima 100 oocysts, E. necatrix 100 oocysts and E. tenella 150 oocysts. In yet another example, a dose comprises a minimum of E. acervulina 139 oocysts, E. maxima 278 oocysts, E. mitis 278 oocysts and E. tenella 417 oocysts. In yet another example, a dose comprises a minimum of E. necatrix 310 oocysts and E. brunetti 155 oocysts.

In some examples, the immunogenic compositions (e.g. vaccines) described herein can include a pharmaceutically acceptable adjuvant. Adjuvants suitable for use in the vaccines may be obtained from any of a number of sources including from natural sources, recombinant sources, and/or be chemically synthesized, etc. They may be obtained from commercial suppliers. Suitable adjuvants for the vaccination of animals include, but are not limited to, Adjuvant 65 (containing peanut oil, mannide monooleate and aluminium monostearate); Freund's complete or incomplete adjuvant; mineral gels, aluminium compounds such as aluminium hydroxide, aluminium phosphate, and alum; surfactants, such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol, and pluronic polyols; polyanions, such as pyran, dextran sulfate, poly IC, polyacrylic acid; peptides, such as muramyl dipeptide, dimethylglycine and tuftsin; oil emulsions; and immune stimulants, such as bacterial and fungal cell wall components (e.g., lipopolysaccarides, lipoproteins, glycoproteins, muramylpeptides, beta-1,3/1,6-glucans), various complex carbohydrates derived from plants (e.g., glycans, acemannan), various proteins and peptides derived from animals (e.g., hormones, cytokines, co-stimulatory factors), and novel nucleic acids derived from viruses and other sources (e.g., double stranded RNA, CpG). In addition, any number of combinations of the aforementioned substances may provide an adjuvant effect, and therefore, can form an adjuvant of the present invention.

Without wishing to be bound by theory, it is thought that the methods described herein are gentler that the traditional methods and are capable of producing a population of oocysts that comprise more viable oocysts and/or a population of oocysts that have a longer shelf life compared to traditional methods. In some examples, the composition comprises at least 10% viable oocysts, at least 20% viable oocysts, at least 30% viable oocysts, at least 40% viable oocysts, at least 50% viable oocysts, at least 60% viable oocysts, at least 70% viable oocysts, at least 80% viable oocysts or at least 90% viable oocysts. Any suitable technique for measuring viability known to the person skilled in the art may be used. In some examples, the composition has a shelf life of at least 12, 18, 24, 30, 36, 42, 48 or 52 weeks when stored under appropriate conditions. In some examples, the composition has a shelf life of at least 12, 18, 24, 30, 36, 42, 48 or 52 weeks when stored at 4-8° C. In some examples, the composition has a shelf life of at least 24, 36, 42, 48 or 52 weeks when stored at 4-8° C. In some examples, the composition has a shelf life of at least 36, 42, 48 or 52 weeks when stored at 4-8° C. In some examples, the composition has a shelf life of at least 24 weeks when stored at 4-8° C. In some examples, the composition has a shelf life of at least 52 weeks when stored at 4-8° C. As used herein, “shelf life” is the amount of time that a composition can be stored and still be suitable for use, for example as a vaccine. Shelf life can be determined using any technique known to the person skilled in the art, for example as described in the Examples hereinbelow. In some examples, the methods described herein are capable of producing a population of oocysts that display less damage to the oocyst compared to traditional methods. Damage to oocysts may be assessed using any technique known to the person skilled in the art, for example techniques for visualising the oocysts. As would be appreciated by the person skilled in the art, shelf life refers to the time post production (i.e. after formulation of the final composition). It is not intended to include the age of the antigens.

In some examples, the compositions described herein (e.g. vaccine compositions) comprise a peroxycarboxylic acid as defined herein. In some examples, the composition (e.g. vaccine composition) comprises peracetic acid. In some examples, the amount of peroxycarboxylic acid present in the composition is sufficient to function as a preservative. For example, the composition (e.g. vaccine composition) comprises less than 1000 ppm, less than 900 ppm, less than 800 ppm, less than 700 ppm, less than 600 ppm, less than 500 ppm, less than 400 ppm, less than 300 ppm, less than 200 ppm or less than peroxycarboxylic acid. In some examples, the composition (e.g. vaccine composition) comprises between about 1 and 500 ppm, between about 1 and 400 ppm, between about 1 and 300 ppm, between about 1 and 200 ppm or between about 1 and 100 ppm peroxycarboxylic acid. In some examples, the vaccine composition comprises between about 10 and 100 ppm peroxycarboxylic acid. In some examples, the vaccine composition comprises between about 1 and 500 ppm, between about 1 and 400 ppm, between about 1 and 300 ppm, between about 1 and 200 ppm or between about 1 and 100 ppm peracetic acid. In some examples, the vaccine composition comprises between about 10 and 100 ppm peracetic acid.

### Uses

The oocysts produced by the methods described herein are suitable for use as a vaccine and/or in vaccine compositions. Compositions comprising Eimeria oocysts find use in methods of immunizing birds against coccidiosis. Methods of vaccinating birds against coccidiosis are known in the art, and include in ovo (e.g., international patent publications WO 96/40234 and WO 96 40233; Pfizer Inc.) and post hatch (e.g., U.S. Pat. No. 3,147,186 to Edgar; U.S. Pat. No. 5,055,292 to McDonald et al.; and U.S. Pat. No. 4,438,097 to Shirley et al.) vaccination methods. Those skilled in the art will appreciate that oocysts may be further processed to release other life stages (e.g., sporozoites or sporocysts) for use in the final vaccine composition, or to produce protozoal proteins for vaccination purposes.

The terms “vaccination” or “immunization” are well-understood in the art. For example, the terms vaccination or immunization can be understood to be a process that increases a subject's immune reaction to antigen, and therefore its ability to resist or overcome infection.

The terms “protective immunity” or “protective immune response,” as used herein, are intended to mean that the host animal mounts an active immune response to the vaccine, such that upon subsequent exposure or a challenge, the animal is able to combat the infection. Thus, a protective immune response will decrease the incidence of morbidity and mortality from subsequent exposure to the pathogen among treated animals. Those skilled in the art will understand that in a commercial animal husbandry setting, the production of a protective immune response may be assessed by evaluating the effects of vaccination on the flock or herd as a whole, e.g., there may still be signs of illness or of morbidity and mortality in a subset of vaccinated animals.

By “active immune response”, it is meant any level of protection from subsequent exposure to the protozoan or protozoan antigens which is of some benefit in a population of subjects, whether in the form of decreased mortality, decreased lesions, improved feed conversion ratios, or the reduction of any other detrimental effect of the disease, and the like, regardless of whether the protection is partial or complete. An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection, or as in the present case, by vaccination. Active immunity can be contrasted with passive immunity, which is acquired through the “transfer of preformed substances (antibody, transfer factor, thymic graft, interleukin-2) from an actively immunized host to a non-immune host.” Id.

In some examples, there is provided a method of immunizing a subject against protozoa infection comprising administering to the subject the vaccine composition described herein. In some examples, there is also provided a method of immunizing poultry against Eimeria infection comprising administering to the poultry the vaccine composition described herein. In some examples, there is provided use of a vaccine composition as described herein for immunizing a subject against protozoa infection. In some examples, there is also provided use of a vaccine composition as described herein for immunizing a subject against Eimeria infection. In some examples, there is provided use of a vaccine composition as described herein for the manufacture of a medicament for immunizing a subject against protozoa infection. In some examples, there is provided use of a vaccine composition as described herein for the manufacture of a medicament for immunizing a subject against Eimeria infection.

In some examples, there is provided a method of enhancing the immune response against an Eimeria parasite in poultry comprising administering the vaccine composition as described herein in an amount effective to enhance the immune response of the poultry to the Eimeria parasite. In some examples, there is provided use of the vaccine composition as described herein in a method of enhancing the immune response against an Eimeria parasite in poultry. In some examples, there is provided use of the vaccine composition as described for the manufacture of a medicament for enhancing the immune response against an Eimeria parasite in poultry. In some examples, the use comprises administering to the poultry in an amount effective to enhance the immune response.

In some examples, there is provided a method of treating or preventing coccidiosis in a poultry, comprising administering to the poultry the vaccine composition described herein in an amount effective to treat or prevent coccidiosis. In some examples, there is provided use of the vaccine composition described herein for treating or preventing coccidiosis in a poultry. In some examples, there is provided use of the vaccine composition described herein for the manufacture of a medicament for treating or preventing coccidiosis in a poultry. In some examples, the use comprises administering to the poultry in an amount effective to treat or prevent coccidiosis.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Examples

The invention disclosed herein will now be further described by reference to the following non-limiting examples.

### Example 1: Identification of Suitable Alternative Sporulation Medium for Eimeria

Production of Eimeria antigen oocysts

Eimeria oocysts of individual species were obtained from Eimeria Pty Ltd's Werribee R&D facility. These included: E. acervulina, E. brunetti, E. maxima, E. mitis, E. necatrix and E. tenella.

Oocysts were produced in isolators in groups of 10-30 birds using regular inocula. Faeces were collected in 24-hour periods during two peak days of production for all species and oocysts were isolated from harvested material using techniques known to the person skilled in the art. Briefly, harvested faecal or caecal material was combined with Reverse Osmosis (RO) water at a 1:10 dilution. The diluted sample was homogenised and a pre-processing oocyst count was performed. The homogenised material was passed through a 500 μm sieve and the filtrate collected. The filtrate were then passed through a 100 μm sieve. The solids caught in the sieves were resuspended with RO water and the sieving procedure repeated. The final sieved material was centrifuged to pellet the solids and the supernatant discarded. The remaining pellet was weighed and resuspended in saturated salt (NaCl) solution at a 1:10 dilution. This solution was centrifuged, the supernatant collected in a clean vessel and combined with RO water at a 1:4 dilution. This solution was again centrifuged and the supernatant discarded. The pellet is resuspended in a small amount of RO water. A final count is performed to determine oocyst yield.

### Preparation of Sporulation Media

The sporulation media was prepared using the candidate chemicals dissolved in RO water at various concentrations as per Table 1.

TABLE 1
Compositions of sporulation media

| Group | Medium composition |
|---|---|
| 1 | 2% w/v potassium dichromate |
| 2 | 0.2% w/v glutaraldehyde |
| 3 | 2% v/v Proxitane (~1000 ppm peracetic acid) |
| 4 | 0.1% v/v Proxitane (~50 ppm peracetic acid) |
| 5 | 2% w/v potassium permanganate |
| 6 | 0.1% w/v potassium permanganate |
| 7 | 1% v/v Virkon S |
| 8 | 0.1% v/v Virkon S |
| 9 | 0.5% v/v hydrogen peroxide |
| 10 | 0.75% v/v hydrogen peroxide, 10% w/v citric |
| | acid and 0.25% v/v propionic acid |
| 11 | 0.4% v/v F10 SC |
| 12 | 1% w/v chloramine B |

### Sporulation in Different Media

For each of the 12 groups of media three replicate experiments were performed on each of the six species listed above. Group 1, 2% potassium dichromate, served as the control against which the other media were measured.

Sporulation was conducted in 250 mL Duran baffled flasks with vented lids. Each replicate was limited to exactly 100 mL in volume, maintaining a solids ratio of 1:10 as much as possible (such that 1:10 was the lowest allowed ratio). The volume of oocyst sample added was limited to a maximum of 10% of the solution based on the concentration of the oocysts to avoid diluting the media. All samples were incubated in a shaking incubator at 27° C. and 150 rpm for 48 hours. Following this the oocysts were washed twice with and re-suspended in PBS-T80 and evaluated for sporulation and oocyst health. The washed samples were stored at 4-8° C.

### Evaluation of Sporulation and Oocyst Health

Samples were evaluated as soon as possible after concluding sporulation. Briefly, the oocyst suspension vessel was mixed thoroughly and a drop of the suspension placed on a glass slide and a cover slip applied. The slide was observed on a microscope under 400× objective. A multi-cell counter was used to count the oocysts. Sporulation level and oocyst health were determined by inspecting at least 100 oocysts. In particular, the percentage of healthy sporulated oocysts for each replicate was determined.

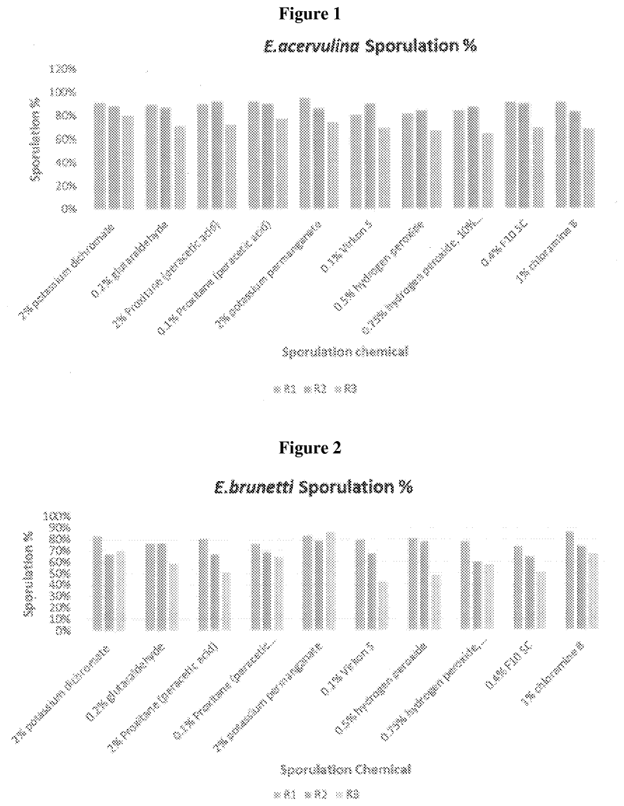

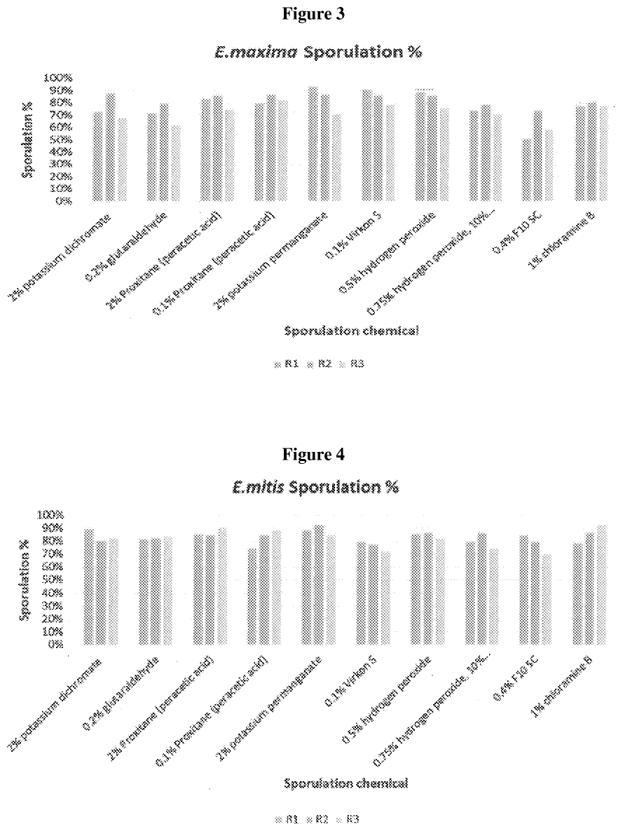

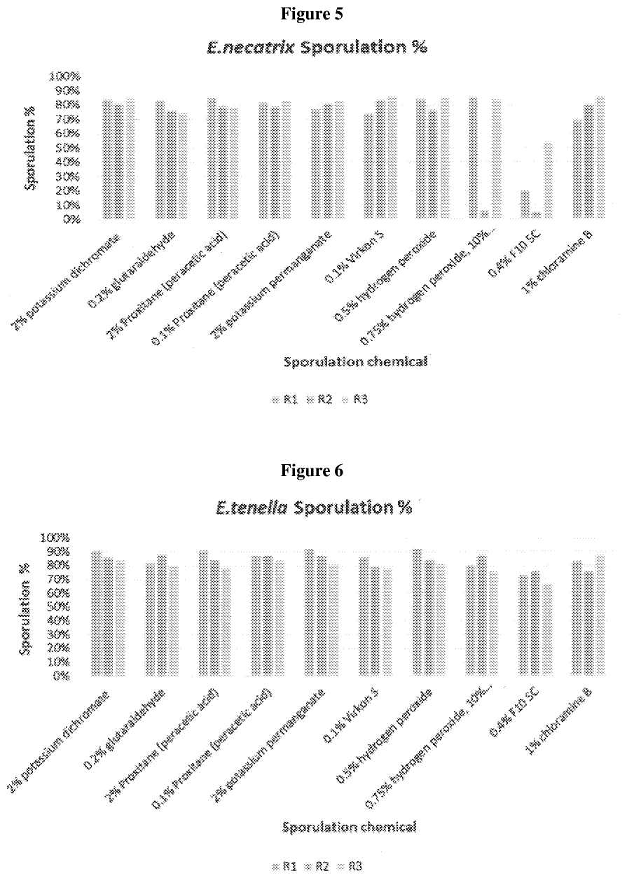

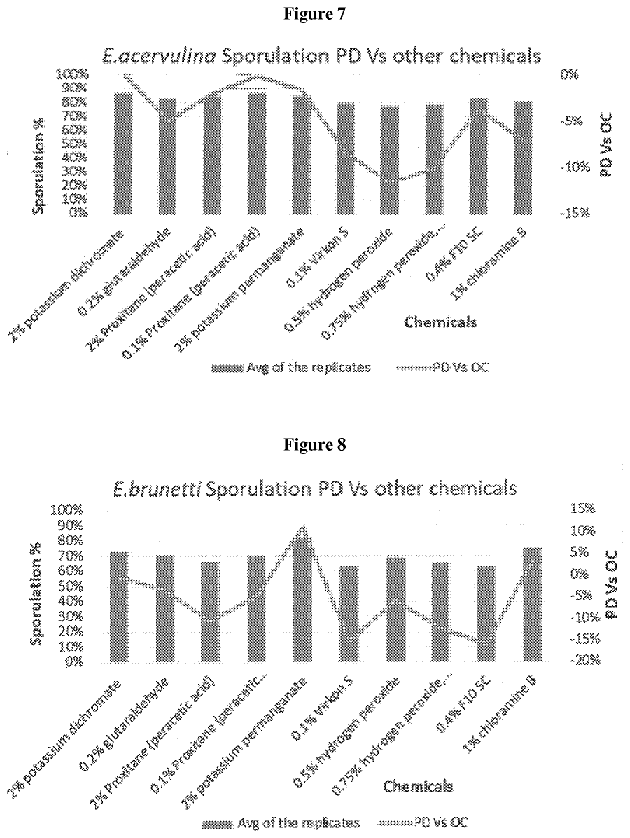

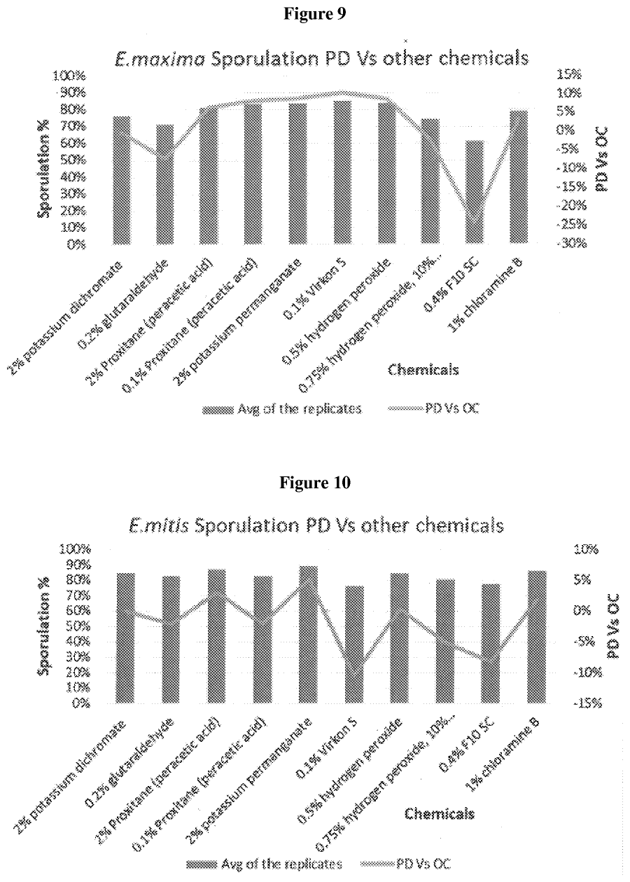

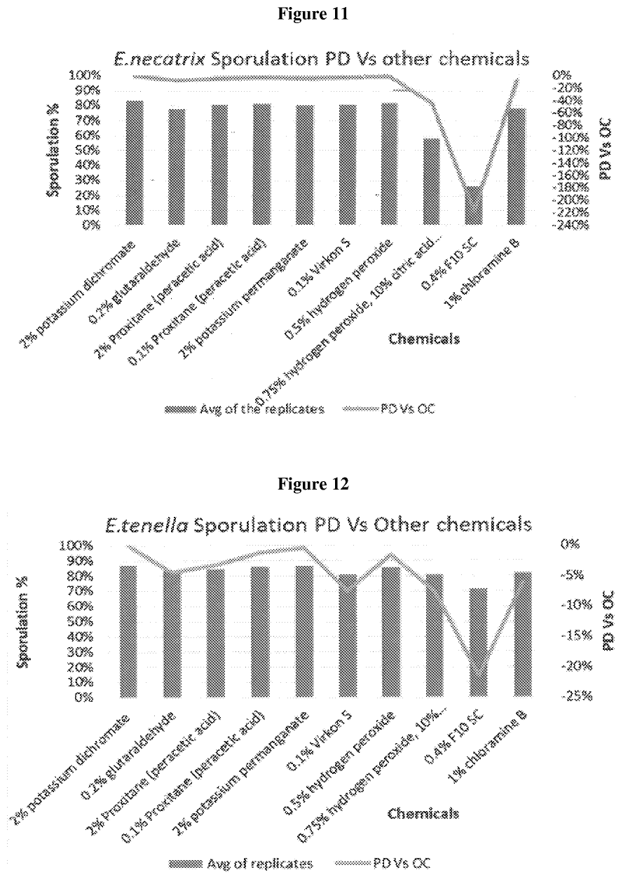

FIGS. 1-6 show the percentage sporulation for each of the sporulation media tested. It is noted that 0.4% F10 SC with E. necatrix failed to yield good sporulation. FIGS. 7-12 show the averaged sporulation from the 3 replicate experiments for each sporulation media tested. This data shows that groups 3, 4, 5 and 9 are comparable to or better than control group 1 in sporulating oocysts across all the species tested. One notable observation was sporulation in E. brunetti treated with group 5 media was considerably higher than the control and the other groups.

Table 2 summarises the average sporulation % across all species for each of the chemical groups.

TABLE 2
Summary of the average of all species in each media

| | Average sporulation % |
|---|---|
| Sporulation media | across all species |
| 2% potassium dichromate | 82% |
| 0.2% glutaraldehyde | 78% |
| 2% Proxitane (~1000 ppm | 81% |
| peracetic acid) | |
| 0.1% Proxitane (~50 ppm | 82% |
| peracetic acid) | |
| 2% potassium permanganate | 85% |
| 0.1% Virkon S | 78% |
| 0.5% hydrogen peroxide | 81% |
| 0.75% hydrogen peroxide, | 73% |
| 10% citric acid and | |
| 0.25% propionic acid | |
| 0.4% F10 SC | 64% |
| 1% chloramine B | 81% |

The total count of oocysts per replicate post sporulation was also calculated and the difference in the oocysts in comparison to the control (group 1) was calculated. To remove staining of the oocysts caused by the potassium permanganate samples from group 5 were treated with 10% metabisulphite at a 1:1 ratio prior to counting. This treatment also cleared all the debris present resulting in a clean sample for counting.

Table 3 summarises the relative difference in oocyst counts compared to the control group 1 (2% potassium dichromate). Overall, no consistent trends towards losses of titre were observed, except for a few outliers that were explained by counting inaccuracy.

TABLE 3
Summary of the relative difference in oocyst counts of the three
replicates of all compared to group 1 (percentage difference)

| | | Counts | | |
|---|---|---|---|---|
| Group | Chemical | R1 | R2 | R3 |
| | E. acervulina | | | |
| 1 | 2% potassium dichromate | 0% | 0% | 0% |
| 2 | 0.2% glutaraldehyde | 1.31 | 0 | −1 |
| 3 | 2% Proxitane (~1000 ppm | 9.70 | −3 | 24 |
| | peracetic acid) | | | |
| 4 | 0.1% Proxitane (~50 ppm | 12.70 | 19 | −4 |
| | peracetic acid) | | | |
| 5 | 2% potassium permanganate | −17.70 | −8 | 4 |
| 8 | 0.1% Virkon S | 9.16 | −1 | −138 |
| 9 | 0.5% hydrogen peroxide | 11.16 | 3 | −298 |
| 10 | 0.75% hydrogen peroxide, | 19.01 | −10 | −151 |
| | 10% citric acid and | | | |
| | 0.25% propionic acid | | | |
| 11 | 0.4% F10 SC | 15.50 | 1 | 11 |
| 12 | 1% chloramine B | 9.43 | 1 | −137 |
| | E. brunetti | | | |
| 1 | 2% potassium dichromate | 0% | 0% | 0% |
| 2 | 0.2% glutaraldehyde | 3 | −22 | 91 |
| 3 | 2% Proxitane (~1000 ppm | −14 | −26 | 76 |
| | peracetic acid) | | | |
| 4 | 0.1% Proxitane (50 ppm | 1 | −45 | 85 |
| | peracetic acid) | | | |
| 5 | 2% potassium permanganate | −1 | −62 | 89 |
| 8 | 0.1% Virkon S | −5 | −17 | 86 |
| 9 | 0.5% hydrogen peroxide | −13 | −14 | 92 |
| 10 | 0.75% hydrogen peroxide, | −7 | −15 | 10 |
| | 10% citric acid and | | | |
| | 0.25% propionic acid | | | |
| 11 | 0.4% F10 SC | 19 | −12 | 87 |
| 12 | 1% chloramine B | −7 | −30 | 91 |
| | E. maxima | | | |
| 1 | 2% potassium dichromate | 0% | 0% | 0% |
| 2 | 0.2% glutaraldehyde | 3 | −21 | −3 |
| 3 | 2% Proxitane (~1000 ppm | −11 | 15 | −105* |
| | peracetic acid) | | | |
| 4 | 0.1% Proxitane (~50 ppm | 11 | 9 | 8 |
| | peracetic acid) | | | |
| 5 | 2% potassium permanganate | −73 | −7 | −2 |
| 8 | 0.1% Virkon S | −12 | 16 | −5 |
| 9 | 0.5% hydrogen peroxide | 5 | 25 | −13 |
| 10 | 0.75% hydrogen peroxide, | −11 | 22 | −10 |
| | 10% citric acid and | | | |
| | 0.25% propionic acid | | | |
| 11 | 0.4% F10 SC | 9 | 0 | −8 |
| 12 | 1% chloramine B | −4 | −2 | −4 |

*counting artefact

| | | | | |
|---|---|---|---|---|
| | E. mitis | | | |
| 1 | 2% potassium dichromate | 0% | 0% | 0% |
| 2 | 0.2% glutaraldehyde | 8.68 | −5 | 1 |
| 3 | 2% Proxitane (~1000 ppm | −282.35* | −42 | 0 |
| | peracetic acid) | | | |
| 4 | 0.1% Proxitane (~50 ppm | 10.74 | −41 | −61 |
| | peracetic acid) | | | |
| 5 | 2% potassium permanganate | −28.14 | −45 | −8 |
| 8 | 0.1% Virkon S | 12.91 | −22 | 4 |
| 9 | 0.5% hydrogen peroxide | 29.90 | −15 | 17 |
| 10 | 0.75% hydrogen peroxide, | −93.24* | −37 | −85 |
| | 10% citric acid and | | | |
| | 0.25% propionic acid | | | |
| 11 | 0.4% F10 SC | −134.43 | −34 | 15 |
| 12 | 1% chloramine B | 12.16 | −25 | 8 |

*Debris stuck to oocyst causing undercounting

| | | | | |
|---|---|---|---|---|
| | E. necatrix | | | |
| 1 | 2% potassium dichromate | 0% | 0% | 0% |
| 2 | 0.2% glutaraldehyde | 7 | 46 | −61.49 |
| 3 | 2% Proxitane (~1000 ppm | 16 | 36 | −9.38 |
| | peracetic acid) | | | |
| 4 | 0.1% Proxitane (~50 ppm | 28 | 31 | −41.00 |
| | peracetic acid) | | | |
| 5 | 2% potassium permanganate | 20 | 33 | −28.83 |
| 8 | 0.1% Virkon S | 33 | 34 | −46.18 |
| 9 | 0.5% hydrogen peroxide | 25 | 37 | −15.15 |
| 10 | 0.75% hydrogen peroxide, | 4 | 18 | −41.84 |
| | 10% citric acid and | | | |
| | 0.25% propionic acid | | | |
| 11 | 0.4% F10 SC | 19 | 13 | −45.29 |
| 12 | 1% chloramine B | 24 | 52 | −27.13 |
| | E. tenella | | | |
| 1 | 2% potassium dichromate | 0% | 0% | 0% |
| 2 | 0.2% glutaraldehyde | 3.12 | 1 | 30.08 |
| 3 | 2% Proxitane (~1000 ppm | 19.44 | −6 | 12.92 |
| | peracetic acid) | | | |
| 4 | 0.1% Proxitane (~50 ppm | 0 | −24 | 39.67 |
| | peracetic acid) | | | |
| 5 | 2% potassium permanganate | −21.51 | −6 | 12.10 |
| 8 | 0.1% Virkon S | −4.44 | 3 | 28.45 |
| 9 | 0.5% hydrogen peroxide | 17.92 | 2 | 26.75 |
| 10 | 0.75% hydrogen peroxide, | −3.08 | 0 | 25.56 |
| | 10% citric acid and | | | |
| | 0.25% propionic acid | | | |
| 11 | 0.4% F10 SC | 3.76 | 5 | 19.74 |
| 12 | 1% chloramine B | −1.16 | 7 | 40.83 |

### Evaluation of Sterility

Oocyst for use in a live coccidiosis vaccine are derived from chicken faeces that are highly laden with microbial load. It is a regulatory requirement to completely ensure that the final product has minimal bioburden levels. Therefore, samples were also tested for sterility (i.e. the ability of the sporulation media to prevent or inhibit growth of microorganisms other than the Eimeria). Briefly, 1 ml of final antigen was incubated with 9 mL of Tryptone Soy Broth (TSB) or 9 ml of Thio broth in an incubator set at 35° C. for 14 days. 1 mL RO water was used in place of the final antigen solution as the negative control. Over the 14 days, vials were checked for turbidity, indicating growth. 100 μL of the final antigen solution was plated on Sabouraud Dextrose Agar (SAB), Horse Blood Agar (HBA) and TSA plates. The SAB plates were incubated aerobically at 25° C. The TSA plates were incubated aerobically at 35° C. or anaerobically at 35° C. using the BE Gaspak container and sachets. The HBA plates were incubated anaerobically at 35° C. using the BE Gaspak container and sachets. Plates are read after 96 hours. Results were assessed as “no growth”, “growth” or “uncertain”. Each replicate contributed 33.33% to sterility for each species treated in each group. The replicate sterility was averaged across the 6 species for each group.

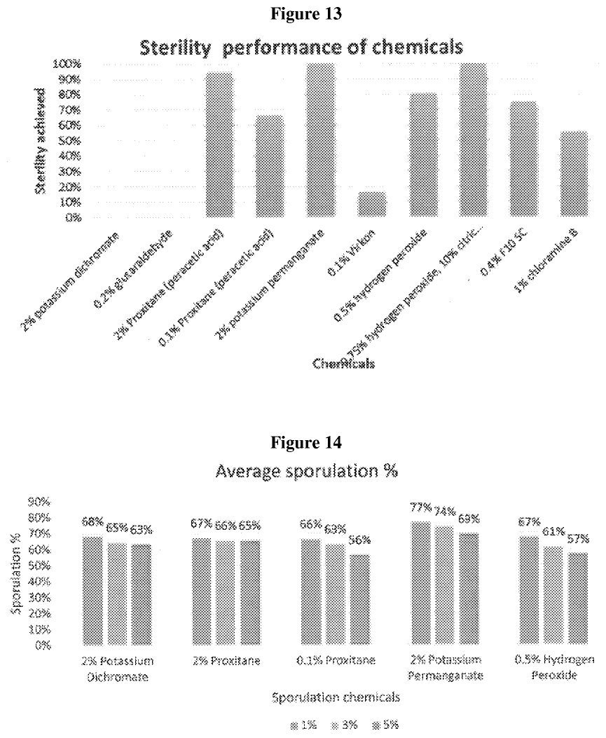

FIG. 13 shows that the control group 1 failed to achieve sterility. Meanwhile, groups 5 and 10 achieved 100% sterility. Group 3 was scored at 94% as the sterility of one of the replicates for E. acervulina was uncertain. Group 9 recorded the next highest level at 80% sterility.

### DISCUSSION

Sporulation and sterilisation are two key important steps in the production of Eimeria vaccines. There is a need for improved methods for that may be used for the manufacture of Eimeria vaccines, for example methods which achieve one or both of sporulation and sterilisation using economical and/or environmentally friendly chemicals. In the present study, twelve different chemicals including the control (Table 1) were tested in triplicate across the six species of Eimeria typically found in Eimeria vaccines. 0.1% potassium permanganate (group 6) and 1% Virkon S (group 7) were found to arrest sporulation and produced the highest numbers of unhealthy oocysts. Given these effects were not observed or reduced at the different concentrations tested (groups 5 and 8), it is believed that the given concentrations resulted in some inhibition of the sporulation of oocyst. These media were not tested further.

The remaining media (groups 2-5 and 8-12) were continued and assessed for sporulation and sterilisation capabilities relative to group 1. Groups 3,4,5 and 9 resulted in sporulation levels that were comparable to or better than the control media. Groups 3, 5 and 10 were highly effective with sterilisation of the oocysts followed by group 9. There were no confirmed titre losses post sporulation, hence it is believed that these chemicals are safe to use for sporulation and sterilisation of oocyst.

### Example 2: Assessment of Sporulation Performance and Bio-Burden Reduction Capabilities of Chemicals at Variable Solids Content

Based on the results of Example 1, Groups 3, 4, 5 and 9 were investigated further. These groups (containing Proxitane, potassium permanganate or hydrogen peroxide) were selected as they appeared capable of sporulating the oocysts and sterilising the sample in a single step.

### Production of Eimeria Antigen Oocysts

Eimeria oocysts of individual species were obtained as described in Example 1.

### Sporulation in Different Media

Sporulation media was prepared as described in Example 1. For each of the five groups of media three replicate experiments were performed for each of E. acervulina, E. brunetti, E. maxima, E. mitis, while one experiment was conducted for both E. necatrix and E. tenella. Group 1, 2% potassium dichromate, again served as the control against which the other media were measured.

Sporulation was conducted in either 50 mL or 15 mL tubes. Each experiment was limited to 2-10 mL in volume, depending on the material available, maintaining a ratio of approximately 1% v/v (a solids ratio of 1:99), 3% v/v (a solids ratio of 3:100) and 5% v/v (a solids ratio of 1:19) solids. Total solids were measured by volume using the gradation on the tubes at the end of Stage 1 isolation of oocysts.

The stage 1 isolated oocysts were distributed proportionately into sporulation tubes. All samples were incubated at 27° C. in a shaking incubator at 150 rpm for 48 hours. Following this the oocysts were washed twice with and re-suspended in PBS-T80. Each replicate was tested for sterility, oocyst count and sporulation percentage as described in Example 1.

### Evaluation of Sporulation and Oocyst Health

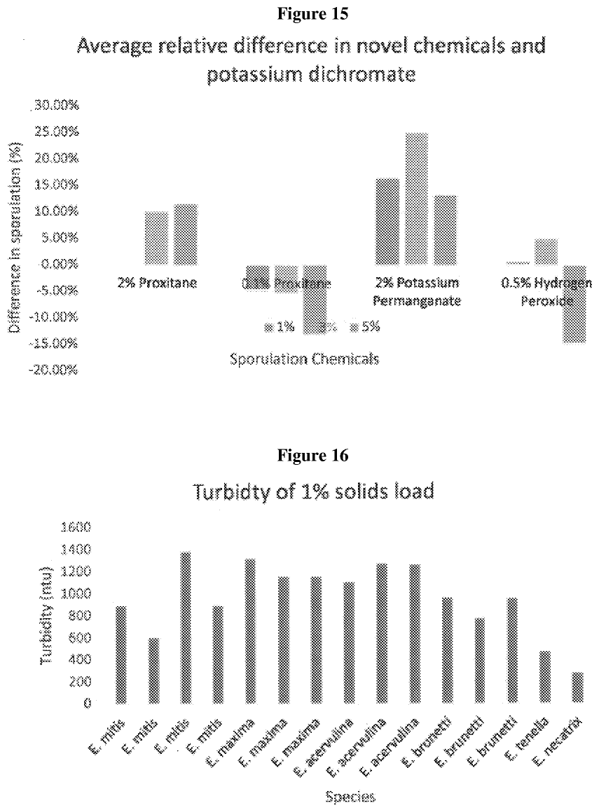

The current study investigated the effective sporulation of oocysts in different chemicals with different solid content. Samples were evaluated as described in Example 1. FIG. 14 shows a clear downward trend in sporulation percentage as the solids content increases, most obvious in the 0.1% Proxitane (~50 ppm peracetic acid), 2% potassium permanganate and 0.5% hydrogen peroxide. Surprisingly, the reduction in sporulation shown by the 2% Proxitane (~1000 ppm peracetic acid) was only 1% as the solid loads increased from 1% to 3% to 5%. This result was better even than the control. When compared to the control, sporulation was better in 2% Proxitane (~1000 ppm peracetic acid) for the 3% and 5% solids content loads by 10.06% and 11.74% respectively. The 1% solids content load was only slightly lower, by 0.11%. While potassium permanganate appears to provide much higher sporulation percentages than dichromate, it was more sensitive to changes in percentage solids content than the 2% Proxitane (~1000 ppm peracetic acid) sample. For 2% potassium permanganate, the reduction in sporulation when the solids content increased from 1% and 5% solids was 8%. In comparison, for 2% Proxitane (~1000 ppm peracetic acid) the reduction in sporulation when the solids content increased from 1% and 5% solids was only 2%. FIG. 15 shows this more clearly by comparing the average relative difference of sporulation percentage in different media compared against the control.

Out of the four sporulation media tested, 2% Proxitane (~1000 ppm peracetic acid) and 2% potassium permanganate faired the best in sporulation, performing as good as or better than dichromate. Potassium permanganate resulted in higher sporulation percentages however some oocysts were damaged or missing. It has been found that unsporulated and unhealthy oocysts are most susceptible to being destroyed and it is thought that the calculated sporulation percentage for potassium permanganate may be biased due to these issues. 0.5% Hydrogen peroxide also achieved good sporulation compared to dichromate except with 5% solids and 0.1% Proxitane (~50 ppm peracetic acid) achieved poorer sporulation on average than dichromate across all solid loads. However, it was ultimately found that hydrogen peroxide produced inconsistent results, particularly when the solids content was varied and/or unpredictable. Hydrogen peroxide is also thought to react quickly. Peracetic acid was found to be more consistent with varying and/or unpredictable solids loads.

### Evaluation of Turbidity and Sporulation

The turbidity of the oocyst preparation used to inoculate the sporulation media was also measured and extrapolated to determine the turbidity of 1%, 3% and 5% solid contents load. Briefly, the turbidity of the oocyst preparation (10% solids) was measured by taking a sample and diluting it 10× or 100×. A 10× dilution would represent 1% solids content load. This was extrapolated to determine the turbidity of the 3% and 5% solid contents loads. In this study, tungsten light turbidometry was measured using Hanna HI98703.

As shown in FIG. 16, the turbidity of the 1% solids content load ranged between 275 ntu and 1380 ntu, with an average of 962 ntu. The E. necatrix sample had the lowest turbidity followed by E. tenella. E. maxima and E. acervulina had a smaller range in measurements, between 1100 ntu and 1310 ntu. The turbidity of E. brunetti was lower but also had little variability, between 770 ntu and 960 ntu. E. mitis had the most variable results in turbidity with the lowest being 600 ntu and the most turbid being 1380 ntu.

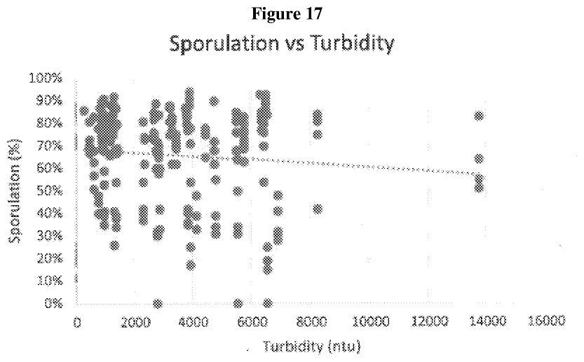

In FIG. 17, sporulation and turbidity were compared for each sporulation media and a slight downward trend is observed as turbidity increases.

### Evaluation of Sterility

The sterility of the replicates was also evaluated as described in Example 1. As expected, the control failed to sterilise the material with the exception of the single E. tenella sample for which results were inconclusive.

2% Proxitane (~1000 ppm peracetic acid) achieved sterility in all trials across all solids contents except for one, an E. maxima sample with 5% solids. 0.1% Proxitane (~50 ppm peracetic acid) had the least success in achieving sterility, with 62% of samples failing sterility. 2% potassium permanganate performed relatively well in achieving sterility with a 19% fail rate. 0.5% hydrogen peroxide had a 21% fail rate with sterility.

This experiment shows that 2% Proxitane (~1000 ppm peracetic acid) is the most reliable at achieving sterility of Eimeria suspensions.

### DISCUSSION

These experiments were performed to identify the threshold at which each chemical was able to sporulate oocysts and also reduce bioburden and achieve sterility. 2% Proxitane (~1000 ppm peracetic acid) was the most successful with only one failed case which was an E. maxima with 5% solid contents load. 2% Potassium permanganate and 0.5% hydrogen peroxide were similar in their numbers; however potassium permanganate and hydrogen peroxide were less predictable. In some instances, a higher solids load passed sterility whereas the lower load did not, raising the question of whether there was possible contamination during the inoculation of samples into TSB. 0.1% Proxitane (~50 ppm peracetic acid) was the least successful in reducing bioburden and failed sterility in most samples. Potassium permanganate was also observed to cause damage to the oocysts, for example peeling of outside layers, which is thought to impact on viability and shelf life.

### Example 3: Assessment of Kinetics of Bacterial and Fungal Inactivation

As demonstrated in the Examples above, 0.5% hydrogen peroxide and 0.1% Proxitane (~50 ppm peracetic acid) may be used to sporulate Eimeria sp., replacing the toxic 2% potassium dichromate currently used in the production of coccidiosis vaccines. These two chemicals, however, were not able to sterilise the oocyst composition and a further addition of Proxitane up a concentration of 2% was used to produce sterile antigens suitable for use in a vaccine. The following study was conducted to help to understand the activity of 0.5% hydrogen peroxide, 50 ppm peracetic acid and 1000 ppm peracetic acid against compendial bacteria and fungi. Each organism was exposed to each chemical mimicking the duration and conditions they would be used with for sporulation or sterilisation. As a control, 0.375% active chlorine sodium hypochlorite with 0.05% Tween-80 at pH 9 was included.

### Preparation of Organisms

Table 4 summarises the bacteria and fungi used for each group that were sourced from Bioproperties QC, Glenorie.

TABLE 4
Test organisms

| | |
|---|---|
| Organism | Cells/spores |

| | | |
|---|---|---|
| A | Staphylococcus aureus ATCC 6538 | Cells |
| B | Pseudomonas aeruginosa ATCC 9027 | Cells |
| C | Bacillus subtilis ATCC 6633 | Spores |
| D | Candida albicans ATCC 10231 | Cells |
| E | Aspergillus brasiliensis ATCC 16404 | Spores |
| F | Clostridium sporogenes ATCC 11437 | Spores |
| G | Paenibacillus rigui | Spores |
| H | Paenibacillus glucanolyticus | Spores |

Organism suspensions were prepared by sub-culturing onto an appropriate growth plate and incubating under standard conditions. The growth was harvested and suspended in appropriate media. The starting titre for most organisms once inoculated into the test media was approximately ≤10⁶ CFU/mL.

### Preparation of Test Chemicals

Solutions of 0.5% v/v hydrogen peroxide, 0.1% v/v Proxitane (~50 ppm peracetic acid) and 2% v/v Proxitane (~1000 ppm peracetic acid) in RO water were prepared prior to use.

### Inactivation of Samples

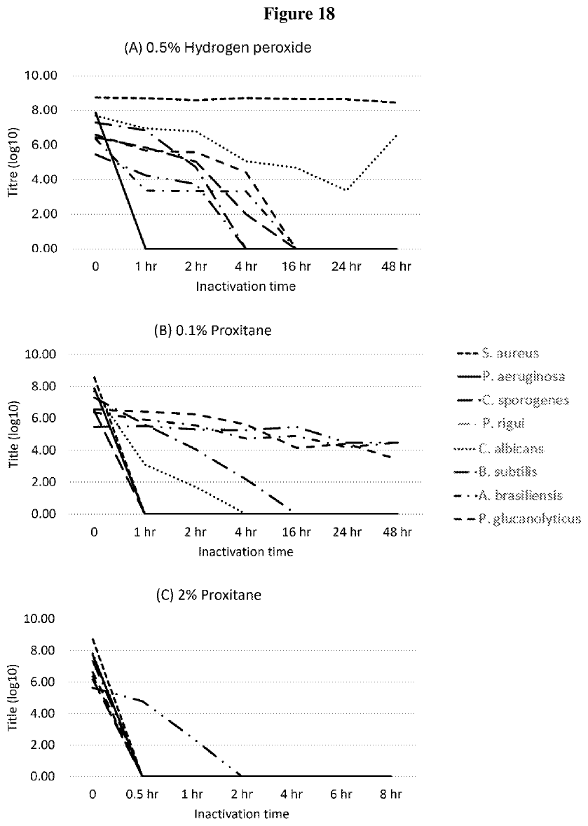

0.1 mL of the harvested bacterial or fungal suspension was added to 9.9 mL of the solution being tested in a sterile glass vial (McCartney container). The inoculated solutions were incubated at 27° C. Zero hours samples were done with sterile water and not the solution being tested to determine a baseline for log reduction calculations. At the appropriate time point, 1 mL of sample was combined with 9 mL of sodium thiosulfate neutralizer (the 10:1 dilution). Serial dilutions in Peptone water (PEP) from 10⁻² to 10⁻⁶ were plated out onto either TSA (bacterial) or SDA (fungal) plates. The 10⁻¹ dilution in neutraliser was also plated. Plates were incubated according to the organisms' requirements and the number of colonies counted at the end of the incubation period. The inactivation kinetics (titre) of each solution tested for each organism were plotted against time and are shown in FIG. 18.

FIG. 18A shows inactivation for 0.5% hydrogen peroxide. The rate varied with some organisms needing more time than others to be eliminated. P. aeruginosa was inactivated within the first hour of treatment but most organisms took longer. Interestingly, S. aureus and C. albicans were still viable after the 48 hours. S. aureus seemed to have not been affected at all with the counts remaining similar across all timepoints whereas C. albicans shows a downward trend, down by 4.32 logs, before going back up at the last time point. It may be possible that the 0.5% hydrogen peroxide had been exhausted after 24 hours allowing the remaining yeast to proliferate again.

FIG. 18B shows that inactivation of organisms by 0.1% Proxitane (~50 ppm peracetic acid) is also quite varied. Five organisms were eliminated within 16 hours, three of which showed no growth at the 1-hour timepoint.

FIG. 18C shows inactivation by 2% Proxitane (~1000 ppm peracetic acid) was much more efficient, all organisms except P. rigui failed to grow on plates by the first timepoint of 30 minutes. P. rigui was inactivated after the 1 hour time point with no plate growth at 2 hours.

### DISCUSSION

Based on these results, 2% Proxitane (~1000 ppm peracetic acid) shows the best inactivation. 0.5% hydrogen peroxide and 0.1% Proxitane (~50 ppm peracetic acid), although effective against some organisms, appear to reach exhaustion for multiple organisms and allows them to survive and maybe even regrow. These results confirmed that 0.5% hydrogen peroxide and 0.1% Proxitane (~50 ppm peracetic acid) were not sufficient in reducing bioburden for all organisms. The 2% Proxitane (~1000 ppm peracetic acid) was very efficient with all organisms inactivated within 30 minutes and P. rigui within 2 hours.

### Example 4: Identification of Suitable Detergent for Sporulation in 2% Proxitane (~1000 ppm Peracetic Acid)

Use of peracetic acid, particularly at 1000 ppm, in the sporulation and sterilisation media was found to be associated with the sticking of debris to oocysts and the sticking of oocysts to each other (e.g. aggregation). The use of a detergent to improve the homogeneity of the Eimeria suspension and reduce sticking when treated with Proxitane was investigated. Initial experiments investigated the effect of Tween 80, TritonX 100, SDS, CTAB and urea (a chaotropic detergent). Follow up experiments investigated the effect of the anionic detergents Sarkosyl, Docusate and ALS.

### Production of Eimeria Antigen Oocysts

Eimeria oocysts of individual species were obtained as described in Example 1.

Preparation of Sporulation Media with Detergents

The 2% Proxitane (~1000 ppm peracetic acid) sporulation media was prepared as described in Example 1. Detergents were added to the 2% Proxitane (~1000 ppm peracetic acid) solution at two concentrations: 0.05% solution and 0.5% solution. A 30% solution of Sarkosyl in 2% Proxitane (~1000 ppm peracetic acid) was prepared first before diluting to the desired concentration. Docusate dissolved in water with heat (up 90° C.) before adding the Proxitane.

### Sporulation in Detergent Media

Oocysts were sporulated in 15 or 50 mL tubes with at least 80% headspace (i.e. 10 mL liquid in 50 mL tube max). The amount of solids (unsporulated oocysts+debris) in this experiments was set to 2%, i.e. a solids ratio of 1:49.

All samples were incubated at 27° C. and about 150 rpm in the shaking incubator for 48 hours. Following sporulation, samples were either stored at 4-8° C. or evaluated immediately.

### Evaluation of Sporulation and Oocyst Health

Sporulation level and oocyst health was evaluated as described in Example 1.

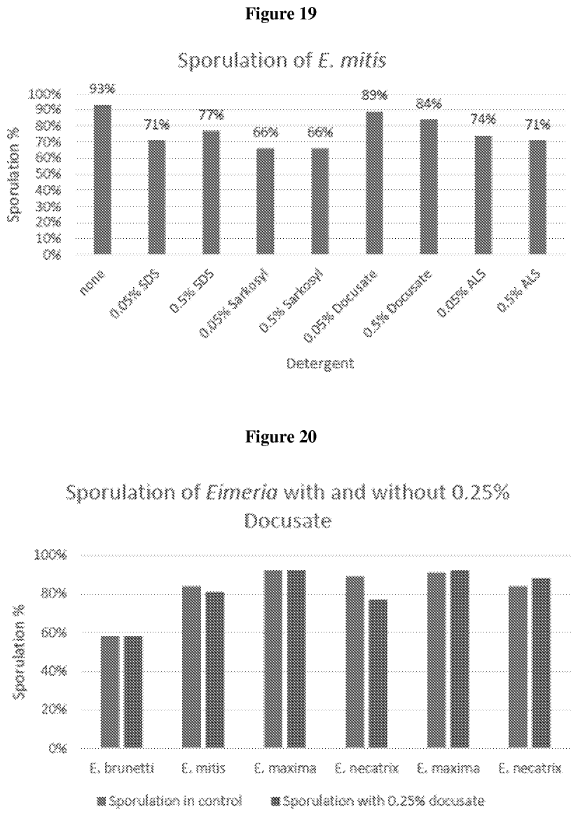

In initial experiments using E. maxima, only SDS was found to reduce sticking (data not shown). However, SDS was also observed to impact percent sporulation, with percent sporulation dropping to 40% at the higher concentration of 0.5%, and cause damage to the oocyst wall upon prolonged storage. Consequently, three more anionic detergents were explored as a follow up trial using E. mitis. Again, no sticking was observed however sticking of debris to oocyst walls was found in the 0.05% sarkosyl and 0.05% docusate groups (FIG. 19). Since docusate did not affect sporulation as much as the other detergents tested, it was further explored at 0.25% concentration. Only one replicate, E. maxima, had sticking of debris in the control. In this instance, the docusate appeared to prevent sticking. Use of 0.25% docusate did not appear to negative impact sporulation. Compared the control, the average relative difference was-2%. The largest difference was observed for a E. necatrix replicate with a relative decrease of 13% (FIG. 20).

### DISCUSSION

Based on the results of this study, 0.25% docusate was added to the sporulation and/or sterilisation media to reduce sticking during sporulation in Proxitane.

### Example 5: Assessment of Eimeria vaccine viability (part 1)

The viability of the oocyst is important for the manufacture of Eimeria vaccines as it contributes to the shelf life of the vaccine. In addition, the protozoa should be viable to confer high level of immunity. Pilot studies demonstrated that 0.1% proxitane and 0.5% hydrogen peroxide were similar to potassium dichromate at producing oocysts which remained viable for 34 weeks for MMAT (E. acervulina 100 sp.oocysts/dose, E. maxima 200 sp.oocysts/dose, E. mitis 200 sp.oocysts/dose, E. tenella 300 sp.oocysts/dose) and 30 weeks for NB (E. brunetti 100 sp.oocysts/dose, E. necatrix 200 sp.oocysts/dose). However, using 2% proxitane as the sporulation media resulted in a reduced shelf life. These studies did not investigate the impact of an additional sterilisation step with 2% proxitane on oocyst viability. In this example, the viability of oocysts produced using the sporulation and sterilisation conditions provided in Table 5 was investigated.

TABLE 5
Sporulation, sterilisation and storage conditions for viability study

| | | | Antigen |
|---|---|---|---|
| | | | Storage |
| Group | Sporulation | Sterilisation | medium |
| A | 2% dichromate | Sodium hypochlorite | RO water + |
| | for 48 h | (0.375% chlorine, pH 9) | 0.05% T80 |
| | | RT for 20 minutes. | |
| B | 0.1% Proxitane | An additional 2% | RO water + |
| | (~50 ppm | proxitane (~1000 | 0.05% T80 |
| | peracetic acid) | ppm peracetic acid) | |
| | for 44 h | for 4 h (continuing | |
| | at 27° C. | sporulation at 27° C.) | |
| C | 0.5% hydrogen | 2% proxitane (~1000 | RO water + |
| | peroxide for 46 h | ppm peracetic acid) | 0.05% T80 |
| | at 27° C. | for 2 h (continuing | |
| | | sporulation at 27° C.) | |

### Production of Eimeria Antigen Oocysts

Eimeria oocysts of individual species were obtained as described in Example 1. The individual Eimeria species were E. acervulina, E. maxima, E. mitis, E. tenella, E. brunetti and E. necatrix.

### Preparation of Sporulation Media

Sporulation media was prepared as described in Example 1.

### Sporulation, Sterilisation and Storage

The oocysts (antigens) used in this Example were sporulated in three different sporulation media as listed in Table 5. All the oocysts were sporulated with sporulation media at 1% suspended solids by turbidimetry (950 NTU) with a minimum of 80% head space and incubated for 48 h at 27° C. Sporulation was performed in either plastic or glass vessels without venting.

Antigens sporulated in 2% potassium dichromate were then treated at room temperature (15-25° C.) with hypochlorite pH-9 with 0.05% Tween-80 (Hypo pH 9-T80) for 20 min at a ratio of solids to liquid of 1:25. Following sterilisation, the antigen was washed twice into RO-water with 0.05% Tween-80 (RO-T80) and the antigen stored in RO-T80 at 2-8° C. before formulating into vaccine.

Antigens sporulated in 0.1% Proxitane (~50 ppm peracetic acid) were sterilised by adding concentrated Proxitane aseptically to make the total concentration 2.1% at 44 h of sporulation (i.e. an additional 2% proxitane was added). The sample was mixed well before being incubated for another 4 h at 27° C. to complete the 48 h sporulation time. Following sterilisation, the antigen was washed twice into RO-water with 0.05% Tween-80 (RO-T80) and the antigen stored in RO-T80 at 2-8° C. before formulating into vaccine.

Antigens sporulated using 0.5% hydrogen peroxide were sterilised by adding concentrated Proxitane aseptically to make the total concentration to 2% at 46 h of sporulation. The sample was mixed well before being incubated for another 2 h at 27° C. to complete the 48 h sporulation time. Following sterilisation, the antigen was washed twice into RO-water with 0.05% Tween-80 (RO-T80) and the antigen stored in RO-T80 at 2-8° C. before formulating into vaccine.

The antigens were counted and volume-adjusted to achieve concentration between 2.50×10⁵ and 1.00×10⁶ sporulated oocysts per mL.

Final antigens were tested for sterility using TSB and Thioglycolate broths as described in Example 1.

### Evaluation of Viability

The antigens were formulated into MMAT and NB vaccines using sterile RO-T80 and tested for viability using procedures known to the person skilled in the art.

### Inoculation and Viability Assessment

The chickens were reared in the isolators and each group was limited to 10 birds that are 7 days old on the day of inoculation. All the birds were tested for being Eimeria free by testing a small faecal sample from each isolator on the day of inoculation. All the pre-inoculation counts were done at Eimeria Development Laboratory (EDL) to track any losses over time. Each chicken was given a dose of 25 L by eye drop for the respective groups involved. The plastic sheet was changed on day 5 and faeces collected on days 6, 7 and 8. The faecal samples were tested for oocyst excretion. All the birds were euthanised on day 8 post-inoculation. Total oocyst output was assessed in the faecal collections and the results for 0.1% proxitane (Group B) and 0.5% hydrogen peroxide (Group C) were compared against Group A, which prepared using dichromate/hypochlorite (i.e. the current vaccine manufacture process).

### Results

The achieved antigen shelf life is summarised in Table 6.

TABLE 6
Viability and shelf life of different sporulation, sterilisation
and storage conditions for the Eimeria species tested
(Achieved antigen shelf life, weeks (species output > threshold)

| | Group A | Group B | Group C |
|---|---|---|---|
| | Dichromate + | 0.1% | 0.5% |
| Eimeria species | Hypochlorite | Proxitane | H₂O₂ |
| E. acervulina | 41 | 42 | 40 |
| E. maxima | 43 | 41 | 32 |
| E. mitis | 39 | 42 | 40 |
| E. tenella | 43 | 41 | 38 |
| E. brunetti | 28 | 30 | 20 |
| E. necatrix. | 12 | 27 | 12 |
| Average all | 34 | 37 | 31 |

Based on the results obtained from this study peracetic acid (Proxitane) was the preferred media for sporulation.

### Example 6: Assessment of Eimeria Vaccine Viability (Part 2)

In this example, the shelf life of vaccines produced using the methods described herein were investigated.

### Production of Eimeria Antigen Oocysts

Eimeria oocysts of individual species were obtained as described in Example 1. The individual Eimeria species were E. acervulina, E. maxima, E. mitis and E. tenella.

### Preparation of Sporulation Media

Sporulation media was prepared as described in Example 1 with the exception that 0.25% docusate is included with 0.5% Proxitane. Briefly, a solution of 0.5% docusate in RO water was prepared. Once the material is pelleted and the solid content is calculated, the pellet was resuspended in RO water to avoid foaming and then combined 1:1 with RO water+0.5% docusate to obtain a suspension with 0.25% docusate and contain a 5% solids content. To this solution concentrated Proxitane was added to make the final concentration of Proxitane to 0.5% (v/v).

### Sporulation in Media

Sporulation took place in the same type of flask for all groups (for example, a 250 mL Duran baffled flask) equipped with a vented lid to prevent pressure build-up from gas production in the media. Each replicate had a volume of 30-100 mL. All samples were incubated at 27° C. in a shaking incubator at 150 rpm for 48 hours. After 48 hours, the sterilization step followed either immediately or after 24 h delay during which the medium was kept at 4-8° C. in a programmable incubator.

### Sterilisation of Antigens

At the end of sporulation an additional 2% Proxitane was added (~1000 ppm peracetic acid) and the antigens incubated for 24 hour at 27° C. as the final sterilisation step. This is achieved by topping up the sporulation media with stock Proxitane to add 2% fresh Proxitane (irrespective of any residual Proxitane persisting from the sporulation step).

### Processing of Antigens

At the end of sterilisation, the antigens were washed once into RO water with 0.05% Tween-80 (T-80) using the centrifuge at 2500 rpm for 10 minutes. They were then resuspended into PBS with 0.05% T-80 and stored at 4-8° C. 1 mL of the final sample used for counting and manual oocyst health checks (OHC). These vaccines were formulated with antigens that were at least 10 weeks old.

### Evaluation of Sporulation and Oocyst Health

Sporulation level and oocyst health was be determined by inspecting at least 100 oocysts under microscope with 400× magnification as described in Example 1. The oocyst count was be conducted before sporulation, after sporulation and sterilisation for each sample to make sure that the concentration of oocysts did not change significantly and to observe any possible clumping. The percentage of healthy sporulated oocysts was determined.

### Evaluation of Bioburden

Bioburden of the samples was assessed before sporulation. The samples were diluted between 10³ to 10¹⁰ fold using peptone water and a 100 μl were plated on TSA and SAB plates before sporulation to assess the bacterial and fungal load.

### Evaluation of Antigen Sterility

Final antigens were tested for sterility using TSB and Thioglycolate broths as described in Example 1.

### Evaluation of Viability

The antigens produced during this study were formulated into MMAT vaccines and tested for viability using procedures known to the person skilled in the art. All formulations were formulated in sterile PBS-T80. Vaccine formulation titre was set to within the approved release titre range as per the manufacture (MMAT: E. acervulina 139 sp.oocysts/dose, E. maxima 278 sp.oocysts/dose, E. mitis 278 sp.oocysts/dose, E. tenella 417 sp.oocysts/dose). The antigens of age 8-12 weeks were formulated into duplicates, namely MMAT 1 and MMAT 2.

### Inoculation and Viability Assessment

The chickens were reared in negative pressure isolators and each group was limited to 10-24 birds that were 7-15 days old on the day of inoculation. All the birds were tested for being Eimeria free by testing a small faecal sample from each isolator on the day of inoculation. All the pre-inoculation counts were done at EDL to track any losses over time. The chickens were grown in two isolators until they are 7-14 days old and transferred to two isolators holding 10 birds each for two subgroups of the vaccine tested. Each chicken was given a dose of 25 μL by eye drop for the respective groups involved. The plastic sheet was changed on day 5 and faeces collected on days 6, 7 and 8. The faecal samples were tested for oocyst excretion. All the birds were euthanised on day 8 post-inoculation.

### Results

In vitro potency assessment: Microscopic and counting confirmed the number of oocysts met batch release criteria from T=0 to up to T=52 weeks for MMAT. All the vaccine batches at the time of inoculation into birds for RPT were confirmed to be with in the minimum and maximum release titre of 2.9E+04 to 4.4E+04 for a 1000 dose vial.

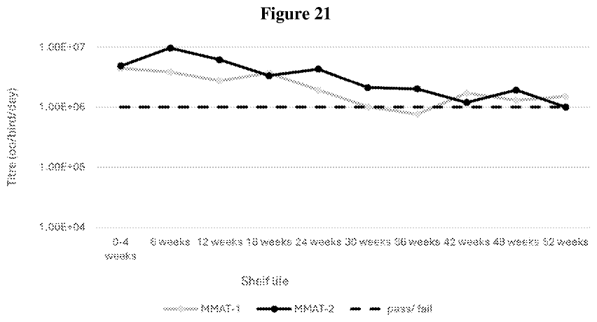

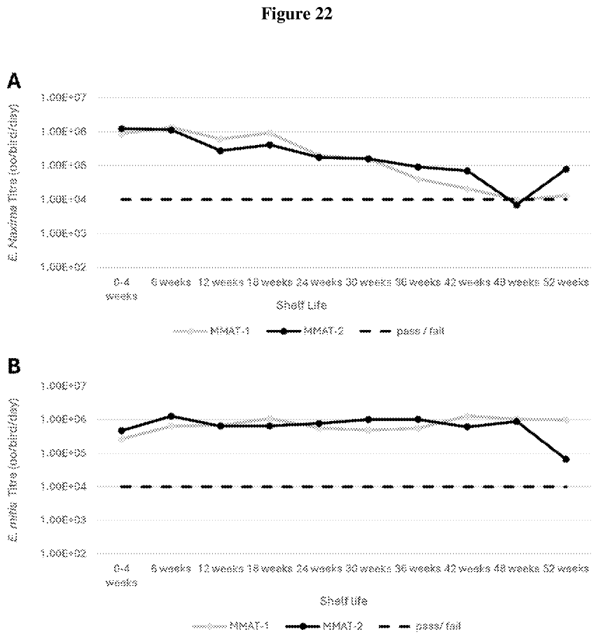

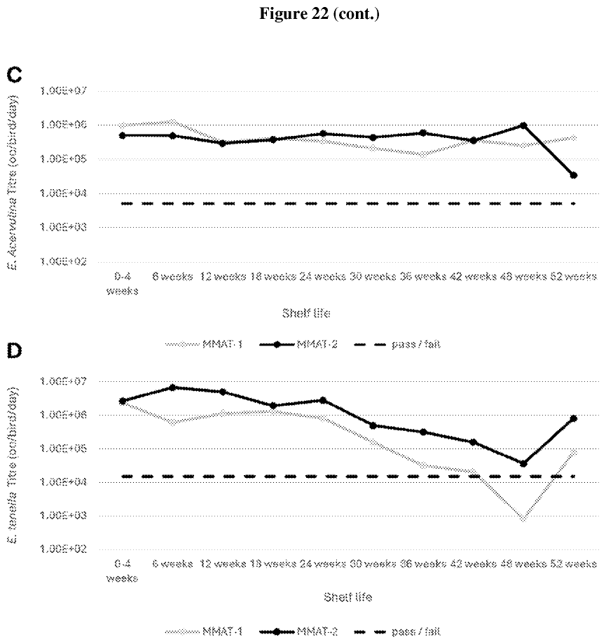

RPT test results: All vaccine batches were subjected to Rapid Potency Test (RPT) testing and met the criteria for RPT. None of the birds showed any abnormal clinical signs or died as a result of vaccination. The average faecal count was greater than 1E+06 excreted oocyst per bird per day and all the vaccine species for MMAT (E. acervulina, E. maxima, E. mitis and E, tenella) were confirmed by qPCR to be present above the threshold levels. The results of the RPT are summarised in Table 7. The summary of the shelf life obtained for MMAT is presented in FIGS. 21 and 22.

TABLE 7
Summary of RPT test results for MMAT Vaccines

| | | Test time | | Microscopic | | E. acervulina | | E. maxima |
|---|---|---|---|---|---|---|---|---|
| Vaccine | Date of | point | Pass/ | (oo/bird/day) | Pass/ | Antigen age | E. acervulina | Antigen age |
| batch | manufacture | (weeks) | Fail | ≥1 × 10⁶ | Fail | (weeks) | ≥5 × 10³ | (weeks) |
| MMAT-1 | 7 Aug. 2023 | 0.3 | Pass | 4.47E+06 | Pass | 11.9 | 9.96E+05 | 12.7 |
| | | 6.3 | Pass | 3.87E+06 | Pass | 17.9 | 1.25E+06 | 18.7 |
| | | 12.3 | Pass | 2.75E+06 | Pass | 23.9 | 3.09E+05 | 24.7 |
| | | 17.3 | Pass | 3.72E+06 | Pass | 28.9 | 4.06E+05 | 29.7 |
| | | 23.3 | Pass | 1.91E+06 | Pass | 24.9 | 3.37E+05 | 35.7 |
| | | 30.1 | Pass | 1.01E+06 | Pass | 41.7 | 2.09E+05 | 42.6 |
| | | 36.1 | Pass | 7.60E+05 | Fail | 47.7 | 1.36E+05 | 48.6 |
| | | 42.3 | Pass | 1.70E+06 | Pass | 53.9 | 3.58E+05 | 54.7 |
| | | 48.3 | Pass | 1.29E+06 | Pass | 59.9 | 2.51E+05 | 60.7 |
| | | 52.1 | Pass | 1.50E+06 | Pass | 63.7 | 4.24E+05 | 64.6 |
| MMAT-2 | 24 Oct. 2023 | 1.1 | Pass | 4.89E+06 | Pass | 11.7 | 4.97E+05 | 8.0 |
| | | 6.1 | Pass | 9.73E+06 | Pass | 16.7 | 4.90E+05 | 13.0 |
| | | 12.1 | Pass | 6.21E+06 | Pass | 22.7 | 2.92E+05 | 19.0 |
| | | 19.0 | Pass | 3.38E+06 | Pass | 29.6 | 3.71E+05 | 25.9 |
| | | 25.0 | Pass | 4.34E+06 | Pass | 35.6 | 5.62E+05 | 31.9 |
| | | 31.1 | Pass | 2.11E+06 | Pass | 41.7 | 4.38E+05 | 38.0 |
| | | 37.1 | Pass | 2.01E+06 | Pass | 47.7 | 5.84E+05 | 44.0 |
| | | 42.1 | Pass | 1.19E+06 | Pass | 52.7 | 3.51E+05 | 49.0 |
| | | 48.1 | Pass | 1.91E+06 | Pass | 58.7 | 9.76E+05 | 55.0 |
| | | 52.1 | Pass | 1.00E+06 | Pass | 62.7 | 3.40E+04 | 59.0 |

| | | | E. mitis | | E. tenella | | VACCINE |
|---|---|---|---|---|---|---|---|
| Vaccine | Date of | E. maxima | Antigen age | E. mitis | Antigen age | E. tenella | qPCR PASS/ |
| batch | manufacture | ≥1 × 10⁴ | (weeks) | ≥1 × 10⁴ | (weeks) | ≥1.5 × 10⁴ | FAIL |
| MMAT-1 | 7 Aug. 2023 | 8.72E+05 | 10.0 | 2.64E+05 | 10.0 | 2.37E+06 | Pass |
| | | 1.36E+06 | 16.0 | 6.46E+05 | 16.0 | 6.11E+05 | Pass |
| | | 6.10E+05 | 22.0 | 6.85E+05 | 22.0 | 1.15E+06 | Pass |
| | | 9.18E+05 | 27.0 | 1.07E+06 | 27.0 | 1.32E+06 | Pass |
| | | 1.99E+05 | 33.0 | 5.61E+05 | 33.0 | 8.13E+05 | Pass |
| | | 1.56E+05 | 39.9 | 4.90E+05 | 39.9 | 1.56E+05 | Pass |
| | | 4.10E+04 | 45.9 | 5.51E+05 | 45.9 | 3.18E+04 | Fail |
| | | 2.11E+04 | 52.0 | 1.30E+06 | 52.0 | 2.03E+04 | Pass |
| | | 9.86E+03 | 58.0 | 1.03E+06 | 58.0 | 8.15E+02 | Fail |
| | | 1.31E+04 | 61.9 | 9.84E+05 | 61.9 | 7.92E+04 | Pass |
| MMAT-2 | 24 Oct. 2023 | 1.24E+06 | 10.0 | 4.71E+05 | 9.9 | 2.68E+06 | Pass |
| | | 1.14E+06 | 15.0 | 1.27E+06 | 14.9 | 6.83E+06 | Pass |
| | | 2.77E+05 | 21.0 | 6.46E+05 | 20.9 | 4.99E+06 | Pass |
| | | 4.15E+05 | 39.9 | 6.49E+05 | 39.9 | 1.95E+06 | Pass |
| | | 1.76E+05 | 33.9 | 7.77E+05 | 33.7 | 2.83E+06 | Pass |
| | | 1.60E+05 | 40.0 | 1.01E+06 | 39.9 | 5.04E+05 | Pass |
| | | 9.25E+04 | 46.0 | 1.02E+06 | 45.9 | 3.18E+05 | Pass |
| | | 7.14E+04 | 51.0 | 6.10E+05 | 50.9 | 1.57E+05 | Pass |
| | | 7.13E+03 | 57.0 | 8.90E+05 | 56.9 | 3.61E+04 | Fail |
| | | 7.95E+04 | 61.0 | 6.61E+04 | 60.9 | 8.20E+05 | Pass |

### Discussion and Conclusions

Live attenuated Eimeria vaccines introduce a controlled number of oocysts to stimulate species-specific protective immunity. Effective protection requires the inclusion of targeted Eimeria species in the vaccine formulation, ensuring immunity against specific antigens. The vaccine's shelf life is assessed by evaluating the viability of these antigens in each batch.

Currently, no in vitro methods exist to evaluate oocyst viability during production. Instead, long-term viability is periodically confirmed through testing in live birds post-manufacture, using the RPT for MMAT. Live attenuated vaccines should contain sufficient oocysts of each species, allowing reinfection and oocyst cycling after inoculation.

The vaccines used in this study were sporulated and sterilized using the methods described herein (i.e. 0.5% (v/v) Proxitane for sporulation and 2% (v/v) Proxitane for sterilization). The test is only regarded as fail if it fails twice consecutively. Based on this MMAT-1 and MMAT-2 were viable until 52 weeks of age. As shown in table 9, this represents an increase based on the shelf life of vaccines produced using current manufacturing processes (2% potassium dichromate as the sporulation medium and sodium hypochlorite as the sterilization medium).

TABLE 9
Antigen and vaccine ages as per the current registered
shelf life and proposed extended shelf life

| | | Shelf life | | Shelf life | |
|---|---|---|---|---|---|
| | | using current | | using methods | |
| Vaccine | | chemistry (weeks) | | described herein (weeks) | |
| type | Species | Antigen | Vaccine | Antigen | Vaccine |
| MMAT | E. acervulina | 10 | 26 | 8-12 | 52 |
| | E. maxima | 10 | 26 | 8-12 | 52 |
| | E. mitis | 10 | 26 | 8-12 | 52 |
| | E. tenella | 10 | 26 | 8-12 | 52 |

Overall, these results suggest that this chemistry results in a sterile vaccine with an improved shelf life.

1. A method for preparing a formulation comprising viable Eimeria oocysts, the method comprising:
incubating viable oocysts with a sporulating composition comprising a first concentration of peroxycarboxylic acid to produce viable, sporulated Eimeria oocysts; and
incubating the viable, sporulated Eimeria oocysts with a sterilising composition comprising a second concentration of the peroxycarboxylic acid to produce the formulation,
wherein the second concentration of peroxycarboxylic acid is greater than the first concentration.

2. The method of claim 1, wherein the incubating is performed in a single reaction vessel.

3. The method of claim 1, wherein:
a) the first concentration of peroxycarboxylic acid is less than 20,000 ppm, or less than 7200 ppm, or less than 1,000 ppm, or less than 750 ppm, or less than 500 ppm, or between about 250-300 ppm; and/or
b) the second concentration of peroxycarboxylic acid is >about 300 ppm, ≥about 500 ppm, ≥about 750 ppm or ≥about 1,000 ppm.

4. (canceled)

5. The method of claim 1, wherein the peroxycarboxylic acid is a C₂-C₅ alkyl peroxycarboxylic acid or peracetic acid.

6. (canceled)

7. The method of claim 1, wherein the oocysts are incubated with:
a) the sporulating composition at a solids content of between about 0.05% v/v and 10% v/v, or between about 1% v/v and 6% v/v, or between about 4.5% v/v and 5.5% v/v; and/or
b) the sterilising composition at a solids content of between about 0.05% v/v and 10% v/v, or between about 1% v/v and 6% v/v, or between about 4.5% v/v and 5.5% v/v.

8. (canceled)

9. The method of claim 1, the method further comprising: processing the sterilised formulation to substantially remove the peroxycarboxylic acid.

10. The method of claim 9, wherein processing the sterilised formulation to substantially remove the peroxycarboxylic acid comprises buffer exchange by diafiltering the sterilised formulation by one or more steps of tangential flow filtration (TFF).

11. The method of claim 10, wherein the at least one or more steps of TFF comprises using a hollow fiber membrane cartridge.

12. The method of claim 1, wherein the formulation is a sterilised formulation.

13. A composition comprising a sterilised formulation produced by the method of claim 12.

14. The composition of claim 13, wherein the composition:
a) comprises viable Eimeria oocysts for at least 12, 18, 24, 36, 39, 42 or 48 weeks after storage at 4-8° C.;
b) a pharmaceutically acceptable carrier; and/or
c) is a vaccine composition.

15.-17. (canceled)

18. A method of:
i) immunizing poultry against Eimeria infection comprising administering to the poultry the vaccine composition of claim 14;
ii) enhancing the immune response against an Eimeria parasite in poultry comprising administering the vaccine composition of claim 14 in an amount effective to enhance the immune response of the poultry to the Eimeria parasite; or
iii) treating or preventing coccidiosis in a poultry, comprising administering to the poultry the vaccine composition of claim 14 in an amount effective to treat or prevent coccidiosis.

19.-20. (canceled)

21. A method for sporulating viable Eimeria oocysts comprising:
incubating the oocysts with a sporulating composition comprising a first concentration of peroxycarboxylic acid to produce viable, sporulated Eimeria oocysts,
wherein the first concentration of peroxycarboxylic acid comprises less than 20,000 ppm peroxycarboxylic acid.

22. The method of claim 21, wherein:
a) at least one peroxycarboxylic acid is a C₂-C₅ alkyl peroxycarboxylic acid;
b) at least one peroxycarboxylic acid is peracetic acid;
c) the concentration of the peroxycarboxylic acid is between about 200 and 300 ppm;
d) the sporulating composition comprises hydrogen peroxide at a concentration of less than about 5000 ppm, optionally wherein the weight ratio of peroxycarboxylic acid to hydrogen peroxide is from about 1:1 to about 1:10;
e) oocysts are incubated with the sporulating composition at a solids content of between about 0.05% v/v and 10% v/v, or between about 1% v/v and 6% v/v, or between about 4.5% v/v and 5.5% v/v;
f) the composition further comprises a detergent and/or antifoaming agent, optionally wherein the detergent is docusate, further optionally 0.25% (w/v) docusate;
g) the pH of the composition is less than 7.0, for example between 2.0 and 4.0;
h) the oocysts are incubated with the sporulating composition for between about 24 to 72 hrs, or about 40 to 48 hrs;
i) the oocysts are incubated with the sporulating composition at a temperature of between about 20 and 32° C., or about 27° C.; and/or
i) the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof.

23.-27. (canceled)

28. The method of claim 1, wherein the composition further comprises:
a) a detergent and/or antifoaming agent, optionally wherein the detergent is docusate, further optionally 0.25% (w/v) docusate;
b) the pH of the composition is less than 7.0, for example between 2.0 and 4.0;
c) the oocysts are incubated with the sporulating composition for between about 24 to 72 hrs, or about 40 to 48 hr;
d) the oocysts are incubated with the sporulating composition at a temperature of between about 20 and 32° C., or about 27° C.; and/or
e) the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof.

29.-33. (canceled)

34. A composition comprising:
i) Eimeria oocysts and peroxycarboxylic acid at a concentration of less than 20,000 ppm; and/or
ii) a population of sporulated Eimeria oocysts, wherein the composition is sterile and, wherein the sporulated oocysts have been treated with a first concentration of peroxycarboxylic acid and a second concentration of peroxycarboxylic acid in a single vessel, wherein the first concentration of peroxycarboxylic acid is less than 1000 ppm and the second concentration of peroxycarboxylic acid increased relative to the first concentration.

35. The composition of claim 34 i), wherein:
a) at least one peroxycarboxylic acid is peracetic acid;
b) the concentration of the peroxycarboxylic acid is from 1 ppm to about 300 ppm;
c) the concentration of the peroxycarboxylic acid is from 800 ppm to about 1200 ppm;
d) the composition has a solids content about 0.05% v/v and 10% v/v, or between about 1% v/v and 6% v/v, or between about 4.5% v/v and 5.5% v/v;
e) the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof;
f) the Eimeria oocysts comprise unsporulated oocysts; and/or
g) the Eimeria oocysts comprise sporulated oocysts.

36.-42. (canceled)

43. The composition of claim 34 ii), wherein;
a) at least one peroxycarboxylic acid is peracetic acid;
b) the first concentration of peroxycarboxylic acid is from about 40 ppm to about 300 ppm, and/or wherein the second concentration of peroxycarboxylic acid is from about 800 ppm to about 1200 ppm;
c) the composition has a solids content about 0.05% v/v and 10% v/v, or between about 1% v/v and 6% v/v, or between about 4.5% v/v and 5.5% v/v;
d) the Eimeria oocysts are selected from the group consisting of oocysts from Eimeria maxima, Eimeria mitis, Eimeria tenella, Eimeria acervulina, Eimeria brunetti, Eimeria necatrix, Eimeria praecox and combinations thereof;
e) the composition is further processed to substantially remove the peroxycarboxylic acid, preferably wherein the composition is diafiltered using TFF;
f) the composition comprises viable oocysts for at least 12, 18, 24, 30, 36, 42 or 48 weeks after storage at 4-8° C.; and/or
g) the composition is sterile.

44.-49. (canceled)
